(19) United States
(12) Patent Application Publication
(10) Pub. No.: US 2010/0197886 A1
(43) Pub. Date: Aug. 5, 2010

(54) MODIFIED POLYMER COMPLEX, COMPLEX MONOMER, POLYMER COMPLEX, AND REDOX,CATALYST

(75) Inventors: Takeshi Ishiyama, Tsukuba-shi (JP); Hideyuki Higashimura, Tsukuba-shi (JP); Sho Kanesaka, Tsukuba-shi (JP)

Correspondence Address: SUGHRUE MION, PLLC, 2100 PENNSYLVANIA AVENUE, N.W., SUITE 800, WASHINGTON, DC, 20037, US

(73) Assignee: Sumitomo Chemical Company, Limited, Chuo-ku (JP)

(21) Appl. No.: 12/671,066

(22) PCT Filed: Aug. 5, 2008
(86) PCT No.: PCT/JP2008/064387

§ 371 Date: Jan. 28, 2010

(30) Foreign Application Priority Data
Aug. 7, 2007 (JP) 2007-205769
Aug. 7, 2007 (JP) 2007-205774

(51) Int. Cl. C08G 79/00; C07D 233/61

(52) U.S. Cl. 528/395; 548/106

(57) ABSTRACT

A modified polymer complex, which is obtained by intermolecular and/or intramolecular crosslinking of a polymer complex via side chains thereof, wherein the polymer complex is a copolymer of a complex monomer meeting the following conditions (i) to (iii) and a comonomer expressed by the following general formula (1): R⁰²R⁰³═R⁰¹E (The definitions of R⁰¹, R⁰², R⁰³ and E are omitted.)

(i) the complex monomer has two or more transition metal atoms;

(ii) the complex monomer has a polydentate ligand containing three or more coordinating atoms that are coordinately bonded to the transition metal atoms; and

(iii) the polydentate ligand has one or more polymerizable functional groups.

### TECHNICAL FIELD

The present invention relates to a modified polymer complex, a complex monomer, a polymer complex and a redox catalyst.

### BACKGROUND ART

A polynuclear complex means a complex that contains two or more metal atoms as central atoms in one complex (Comprehensive Dictionary on Chemistry, first edition, 1994, Tokyo Kagaku Dozin Co., Ltd.). Since such a polynuclear complex can exert specific and various reactivities based on interactions among plural metal sites, it can be used as a reaction catalyst, and for example, it is useful as a catalyst related to a chemical reaction involving electron transfer such as a redox catalyst (see, for example, Surface 2003, 41 (3), 22 by Ken-ichi Oyaizu and Makoto Yuasa).

For a polynuclear complex, a manganese binuclear complex is known as a catalyst that decomposes hydrogen peroxide into water and oxygen (hydrogen peroxide decomposition catalyst) while suppressing generation of free radicals (hydroxyl radical, hydroperoxy radical, and the like) (see, for example, A. E. Beolrijk and G. C. Dismukes Inorg. Chem. 2000, 39, 3020). Furthermore, a catalyst obtained by heat-treating protein containing metals was reported (see, for example, Japanese Unexamined Patent Publication (JP-A) No. 2004-217507).

However, when the above-described conventional manganese binuclear complex is used as a hydrogen peroxide decomposition catalyst, stability, particularly, heat stability is not sufficient, and use in a heating reaction, and the like causes a problem, and thus, a catalyst more excellent in heat stability has been desired. A catalyst obtained by heat-treating protein containing metals is not only expensive but also has poor storage stability since it is a biological substance, and therefore, a catalyst using the protein as a raw material has difficulty in production reproducibility.

Thus, an object of the present invention is to provide a modified polymer complex that can be used as a catalyst not only having a catalytic ability capable of decomposing hydrogen peroxide into water and oxygen but also being excellent in heat stability, and a catalyst using the same.

### DISCLOSURE OF THE INVENTION

That is, the present invention provides the following aspects.
[1] A modified polymer complex, which is obtained by intermolecular and/or intramolecular crosslinking of a polymer complex via side chains thereof, wherein the polymer complex is a copolymer of a complex monomer meeting the following conditions (i) to (iii) and a comonomer expressed by the following general formula (1):
(i) the complex monomer has two or more transition metal atoms;
(ii) the complex monomer has a polydentate ligand containing three or more coordinating atoms that are coordinately bonded to the transition metal atoms; and
(iii) the polydentate ligand has one or more polymerizable functional groups;

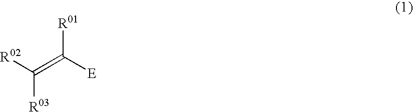

wherein E denotes a cyano group, a carboxyl group, a formyl group, a carbamoyl group, a phosphonic acid group, a sulfonic acid group, a halogeno group, a —CONHCH₂OR⁰⁴ group or a —Si(OR⁰⁵)₃ group, each of R⁰¹, R⁰² and R⁰³ independently denotes a hydrogen atom, a halogeno group, a cyano group, a —COOR⁰⁴ group, an alkyl group having 1 to 10 carbon atoms which may have a substituent, or an aryl group having 6 to 10 carbon atoms which may have a substituent; R⁰⁴ is a hydrogen atom, an alkyl group having 1 to 10 carbon atoms which may have a substituent, or an aryl group having 6 to 10 carbon atoms which may have a substituent; and R⁰⁵ is a hydrogen atom, an alkyl group having 1 to 10 carbon atoms which may have a substituent, or an aryl group having 6 to 10 carbon atoms which may have a substituent.

The modified polymer complex of the present invention is obtained by further crosslinking a polymer complex that is obtained by copolymerizing a complex monomer having a polymerizable functional group and a comonomer (vinyl compound). As described above, since the modified polymer complex of the present invention contains a skeleton derived from a complex monomer, it has a decomposition ability to decompose hydrogen peroxide into water and hydrogen and also exerts a high reaction activity as a redox catalyst. Since crosslinking occurs in the modified polymer complex of the present invention due to a reaction of a side chain of a polymer complex (polymer) obtained by copolymerizing a complex monomer and a commoner, the modified polymer complex is particularly excellent in heat stability and functions as a catalyst that can be used in a reaction at high temperature.
[2] The modified polymer complex according to [1], wherein the transition metal atoms are transition metal atoms in the first transition element series.
[3] The modified polymer complex according to [1] or [2], wherein at least one structure in which two transition metal atoms are coordinately bonded to the same coordinating atom is present.
[4] The modified polymer complex according to [1] to [3], wherein at least one structure in which a coordinating atom that is coordinately bonded to one transition metal atom and a coordinating atom that is coordinately bonded to another transition metal atom are bonded via 1 to 4 covalent bonds is present.
[5] The modified polymer complex according to [1] to [4], wherein the polydentate ligand has a structure expressed by the following general formula (2):

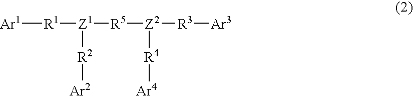

wherein each of groups R¹, R², R³, R⁴ and R⁵ independently denotes a divalent group, and each of Z¹ and Z² independently denotes a nitrogen atom or a trivalent group; and at least one of Ar¹, Ar², Ar³, Ar⁴, R¹, R², R³, R⁴ and R⁵ has a polymerizable functional group.
[6] The modified polymer complex according to [1] to [5], wherein the polydentate ligand has a structure expressed by the following general formula (3a) or (3b):

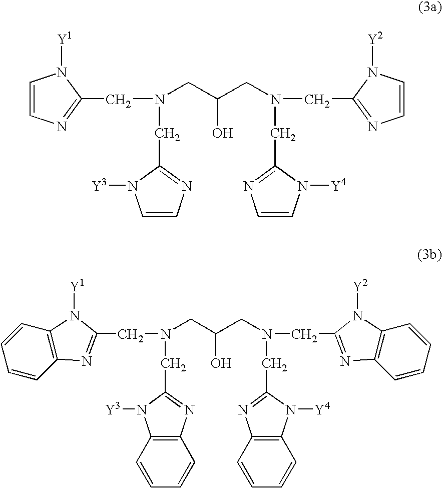

wherein each of Y¹, Y², Y³and Y⁴ independently denotes a hydrogen atom, an alkyl group having 1 to 50 carbon atoms, or an aromatic group having 2 to 60 carbon atoms, and at least one of Y¹, Y², Y³ and Y⁴ is an alkyl group having 1 to 50 carbon atoms which has a polymerizable functional group, or an aromatic group having 2 to 60 carbon atoms which has a polymerizable functional group.
[7] The modified polymer complex according to [1] to [6], wherein the comonomer contains at least one crosslinkable comonomer selected from a comonomer in which E is a cyano group, a comonomer in which E is a formyl group, and a comonomer in which E is a carbamoyl group in the general formula (1).
[8] The modified polymer complex according to [1] to [7], wherein the comonomer contains at least one crosslinkable comonomer selected from the group consisting of acrylonitrile, methacrylonitrile, acrylamide, methacrylamide, and chloroacrylonitrile.
[9] The modified polymer complex according to [1] to [8], wherein the comonomer contains at least one hydrophilic comonomer selected from the group consisting of acrylic acid, methacrylic acid, vinylphosphonic acid, vinylsulfonic acid, styrenesulfonic acid, a styrenesulfonate salt, and a styrenesulfonic acid ester.
[10] The modified polymer complex according to [1] to [9], wherein the comonomer contains at least one of the crosslinkable comonomers and at least one of the hydrophilic comonomers.
[11] The modified polymer complex according to [1] to [10], wherein the modified polymer complex is obtained by copolymerizing the complex monomer and the comonomer in the presence of a carbon additive.
[12] The modified polymer complex according to [1] to [11], wherein the polymer complex shows a molecular ionic peak having m/Z of 53 or 67 when a mass number of a molecular ion is assumed to be m and a charge number of the molecular ion is assumed to be Z in a thermogravimetric-mass spectrum.
[13] The modified polymer complex according to [1] to [12], which is obtained by intermolecular and/or intramolecular crosslinking of the polymer complex by a heat treatment, a radiation irradiation treatment, an electromagnetic wave irradiation treatment or a discharge treatment,

wherein a weight loss after the treatment is 3% by weight or more and 50% by weight or less based on the weight before the treatment.
[14] The modified polymer complex according to [1] to [13], which is obtained by intermolecular and/or intramolecular crosslinking of the polymer complex by a heat treatment at a temperature within the range from 200 to 900° C.
[15] The modified polymer complex according to [1] to [14], which is in a particulate form having an average particle diameter derived from a scanning electron micrograph within the range from 10 nm to 10 μm.
[16] The modified polymer complex according to [1] to [15], wherein a content of the transition metals is 8 to 0.01% by weight in an elemental analysis with an ICP optical emission spectrometry.
[17] The modified polymer complex according to [1] to [16], wherein a peak maximum is shown within the ranges from 1390 to 1440 cm⁻¹ and 1590 to 1630 cm⁻¹ in an infrared spectroscopy.
[18] The modified polymer complex according to [1] to [17], wherein a gTOP defined by the following (Formula 1) is within the range from 1.8000 to 2.2400 in a solid electron spin resonance spectrum:

gTOP=hν/βH (Formula 1)

wherein h denotes a Planck constant, ν denotes a resonant frequency of a measured electromagnetic wave, β denotes a Bohr magneton, and H denotes a magnetic field intensity showing a maximum of an observed ESR signal, respectively.
[19] A complex monomer meeting the following conditions (i′) to (iv′):
(i′) the complex monomer has one or more transition metal atoms;
(ii′) the complex monomer has a polydentate ligand containing three or more coordinating atoms that are coordinately bonded to the transition metal atoms;
(iii′) the polydentate ligand has one or more polymerizable functional groups; and
(iv′) the complex monomer has any structure of an organic acid salt structure, an amine salt structure, an ammonium salt structure, a pyridinium salt structure, an imidazolium salt structure, a hydroxyl group structure, an ether structure, and an acid amide structure.
[20] The complex monomer according to [19], comprising at least one of the functional groups expressed by the following general formulas (1-1), (1-2), (1-3), (1-4), (1-5), (1-6), (1-7), (1-8) and (1-9) in the structure of (iv′):

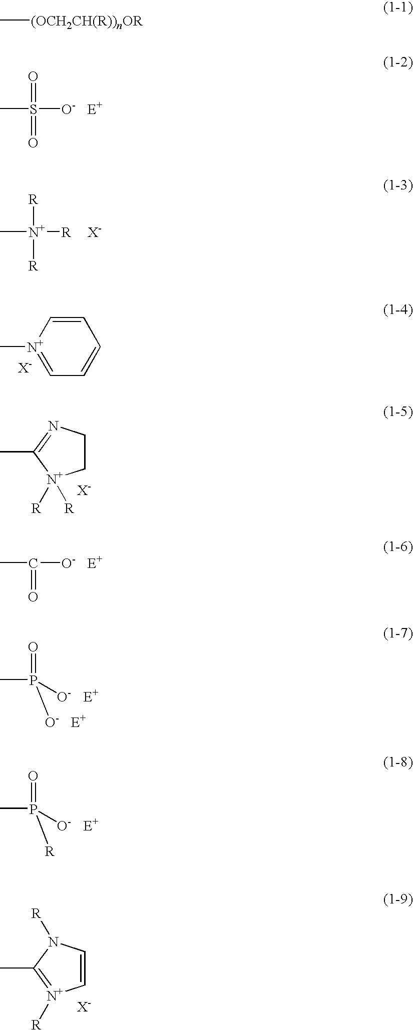

wherein n denotes an integer of 1 to 500, E⁺ denotes a proton, a lithium ion, a sodium ion, a potassium ion, a rubidium ion, a cesium ion or an ammonium ion, R denotes a hydrogen atom, an alkyl group having 1 to 50 carbon atoms which may have a substituent, or an aryl group having 6 to 50 carbon atoms which may have a substituent, and X⁻ denotes a fluoride ion, a chloride ion, a bromide ion, an iodide ion, a methanesulfonate ion, or a trifluoromethanesulfonate ion, respectively.
[21] The complex monomer according to [19] or [20], wherein the transition metal atom is a transition metal atom in the first transition element series.
[22] The complex monomer according to [19] to [21], having a structure expressed by the following general formula (2-1):

(L⁰¹)ₚ(M)ₘ(L⁰²)_{q} (2-1)

wherein M denotes a transition metal atom, m denotes an integer of 1 to 20, p denotes an integer of 1 to 5, and q denotes an integer of 1 to 20, respectively; L⁰¹ is a polydentate ligand having 3 or more atoms including a nitrogen coordinating atom, which has a substituent containing a polymerizable functional group or a functional group expressed by the general formula (1-1); and L⁰² is a ligand or a counter ion, which has a substituent containing a polymerizable functional group or a functional group expressed by the general formula (1-1), provided that a combination of the substituents in L⁰¹ and L⁰² is a combination of a polymerizable functional group and a functional group expressed by the general formula (1-1).
[23] The complex monomer according to [19] to [22], comprising two or more transition metal atoms,

wherein at least one structure in which two transition metal atoms among the two or more transition metal atoms are coordinately bonded to the same coordinating atom is present.
[24] The complex monomer according to [19] to [23], comprising two or more transition metal atoms,

wherein at least one structure in which a coordinating atom that is coordinately bonded to one transition metal atom among the two or more transition metal atoms and a coordinating atom that is coordinately bonded to a transition metal atom other than the one transition metal atom among the two or more transition metal atoms are bonded via 1 to 4 covalent bonds is present.
[25] The complex monomer according to [19] to [24], wherein L⁰¹ in the general formula (2-1) has a structure expressed by the following general formula (2):

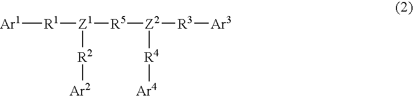

wherein each of Ar¹, Ar², Ar³ and Ar⁴ independently denotes a nitrogen-containing aromatic heterocyclic group, each of R¹, R², R³, R⁴ and R⁵ independently denotes a divalent group, and each of Z¹ and Z² independently denotes a nitrogen atom or a trivalent group, respectively; and at least one of Ar¹, Ar², Ar³, Ar⁴, R¹, R², R³, R⁴ and R⁵ has one polymerizable functional group.
[26] The complex monomer according to [19] to [25], having a structure in which L⁰¹ in the general formula (2-1) is expressed by the following general formula (3a) or (3b):

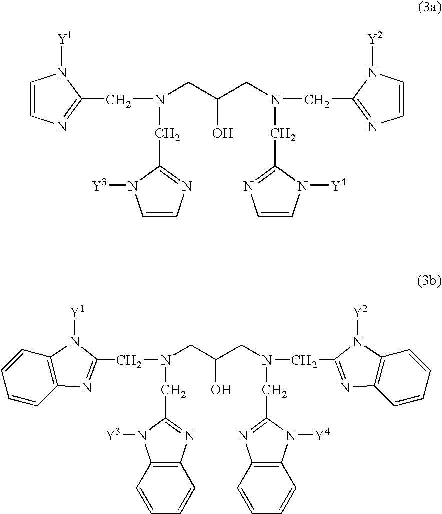

wherein each of Y¹, Y², Y³ and Y⁴ independently denotes a hydrogen atom, an alkyl group having 1 to 50 carbon atoms, or an aromatic group having 2 to 60 carbon atoms, and at least one of Y¹, Y², Y³ and Y⁴ is an alkyl group having 1 to 50 carbon atoms which has a polymerizable functional group, or an aromatic group having 2 to 60 carbon atoms which has a polymerizable functional group.
[27] The complex monomer according to [19] to [26], having a structure in which L⁰² in the general formula (2-1) is expressed by the following general formula (40):

G⁰¹—(OCH₂CH(R))ₙOR (40)

wherein R denotes a hydrogen atom, an alkyl group having 1 to 50 carbon atoms which may have a substituent, or an aryl group having 6 to 50 carbon atoms which may have a substituent, and G⁰¹ denotes a substituent containing a functional group expressed by any of the following general formulas (4-1), (4-2), (4-3) and (4-4), respectively:

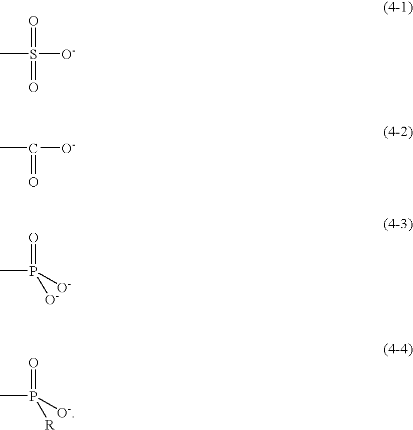

[28] A polymer complex obtained by polymerizing the complex monomer according to [19] to [27].
[29] A polymer complex obtained by copolymerizing the complex monomer according to [19] to [27] and a comonomer.
[30] A redox catalyst, comprising the modified polymer complex according to [1] to [18], the complex monomer according to [19] to [28], or the polymer complex according to [28] or [29].

### BRIEF DESCRIPTION OF THE DRAWINGS

FIG. 1 is a ¹H-NMR analysis chart of a bbpr-CH₂St ligand in Production Example 1.

FIG. 2 is an IR analysis chart of a polymer complex in Production Example 4.

FIG. 3 is an IR analysis chart of a modified polymer complex obtained in Example 1.

FIG. 4 is a graph showing a variation with time of generated oxygen amounts in hydrogen peroxide decomposition tests of modified polymer complexes in Examples 2 and 3.

FIG. 5 is a graph showing a variation with time of generated oxygen amounts in hydrogen peroxide decomposition tests in Comparative Examples 1 and 2.

FIG. 6 is a ¹H-NMR analysis chart of P₄₅C₄Na in Production Example 5.

FIG. 7 is an IR analysis chart of a complex monomer in Example 4.

FIG. 8 is a scanning electron micrograph of a polymer complex obtained in Example 5.

FIG. 9 is an IR analysis chart of the polymer complex obtained in Example 5.

FIG. 10 is a graph showing a variation with time of a tubular furnace temperature in a heat treatment in Example 6.

FIG. 11 is a scanning electron micrograph of a polymer complex carbon black composite obtained in Example 7.

FIG. 12 is a scanning electron micrograph of a modified polymer complex obtained in Example 8.

FIG. 13 is a graph showing a variation with time of a hydrogen peroxide decomposition rate in a hydrogen peroxide decomposition test in Example 61.

FIG. 14 is a scanning electron micrograph of a polymer complex obtained in Example 63.

### BEST MODE FOR CARRYING OUT THE INVENTION

Preferable embodiments of the present invention will be specifically described below; however, the present invention is not limited to the following embodiments.

### (Complex Monomer)

A complex monomer according to the present invention has first and second embodiments.

The complex monomer of the first embodiment will be first specifically described. Hereinafter, the complex monomer of the first embodiment is also simply referred to as the complex monomer 1.

The complex monomer 1 used in synthesis of a modified polymer complex is a polynuclear complex having two or more transition metal atoms, and contains, as a ligand, a polydentate ligand containing three or more coordinating atoms coordinately bonding to the transition metal atoms. In addition, the polydentate ligand has one or more polymerizable functional groups.

Since the complex monomer 1 has a polymerizable functional group, the complex monomer 1 can copolymerize with a comonomer expressed by the general formula (1), and a polymer complex can be obtained due to the copolymerization. The complex monomer 1 can also impart a catalyst activity to a modified polymer complex obtained by crosslinking of the polymer complex because it has a polynuclear complex structure. That is, the modified polymer complex that is a final product is useful as a redox catalyst, and in particular, can be used as a catalyst that decomposes hydrogen peroxide into water and oxygen while suppressing generation of free radicals (hydroxyl radical, hydroperoxy radical, and the like).

The number of transition metal atoms in the complex monomer 1 is preferably 2 or more and 8 or less, more preferably 2 or more and 4 or less, and particularly preferably 2 or 3. The transition metal atoms may be uncharged or charged ions. A plurality of the transition metal atoms contained in the complex monomer 1 may be identical or different from one another.

Specific examples of the transition metal atoms contained in the complex monomer 1 can include transition metal atoms in the first transition element series selected from the group consisting of scandium, titanium, vanadium, chromium, manganese, iron, cobalt, nickel, copper, and zinc; yttrium, zirconium, niobium, molybdenum, ruthenium, rhodium, palladium, silver, cadmium, lanthanum, cerium, praseodymium, neodymium, samarium, europium, gadolinium, terbium, dysprosium, holmium, erbium, thulium, ytterbium, hafnium, tantalum, tungsten, rhenium, osmium, iridium, platinum, gold, mercury, actinium, thorium, protactinium, and uranium.

Among the above-described transition metal atoms, it is preferable to use transition metal atoms in the first transition element series, zirconium, niobium, molybdenum, ruthenium, rhodium, palladium, silver, lanthanum, cerium, praseodymium, neodymium, samarium, europium, gadolinium, terbium, dysprosium, holmium, erbium, thulium, ytterbium, tantalum, tungsten, rhenium, osmium, iridium, platinum, and gold, it is more preferable to use transition metal atoms in the first transition element series, zirconium, niobium, molybdenum, ruthenium, rhodium, palladium, silver, lanthanum, cerium, samarium, europium, ytterbium, tantalum, tungsten, rhenium, osmium, iridium, platinum, and gold, it is further more preferable to use transition metal atoms in the first transition element series, it is particularly preferable to use vanadium, chromium, manganese, iron, cobalt, nickel, and copper, and among these elements, it is more particularly preferable to use manganese, iron, cobalt, nickel, and copper, and it is most preferable to use manganese.

The complex monomer 1 has a polydentate ligand containing 3 or more coordinating atoms coordinately bonding to transition metal atoms, which thus makes it possible to form the stable complex monomer 1 due to a chelate effect of the polydentate ligand. The number of coordinating atoms is 3 or more, preferably 3 or more and 50 or less, further more preferably 5 or more and 25 or less, and particularly preferably 6 or more and 20 or less.

A polydentate ligand in the complex monomer 1 has one or more polymerizable functional groups. The polymer complex 1 and a comonomer are copolymerized with the use of polymerization reactivity of the polymerizable functional groups to form a high molecular weight compound, thereby making it possible to easily induce a nonuniform complex catalyst. The number of polymerizable functional groups contained in the complex monomer 1 is preferably 1 or more and 20 or less, more preferably 2 or more and 10 or less, and particularly preferably 4 or more and 8 or less.

Examples of the polymerizable functional group include a vinyl group, an acrylic group, a methacrylic group, an allyl group, a propenyl group, a butenyl group, a butadinyl group, a styryl group, a 2-vinylbenzyl group, a 3-vinylbenzyl group, and a 4-vinylbenzyl group; a vinyl group, a styryl group, an allyl group, a 2-vinylbenzyl group, a 3-vinylbenzyl group, and a 4-vinylbenzyl group are preferable; and a vinyl group, a styryl group, an allyl group, and a 4-vinylbenzyl group are more preferable.

In the complex monomer 1, it is preferable that at least two transition metal atoms among plural transition metal atoms are closely located in a polynuclear complex molecule.

When two transition metal atoms in a polynuclear complex, that is, the complex monomer 1 are assumed to be M1 and M2, one of coordinating atoms that coordinates to M1 is assumed to be AM1, and one of coordinating atoms that coordinates to M2 is assumed to be AM2, the number of covalent bonds present between AM1 and AM2 can be calculated and used as “an index of closely located transition metal atoms.” Herein, when AM1 and AM2 are directly bonded, the number of covalent bonds is 1, when AM1 and AM2 are covalently bonded on the whole via one atom, the number of covalent bonds is 2, and when AM1 and AM2 are covalently bonded on the whole via n atoms, the number of covalent bonds is (n+1). When atoms are directly bonded with a double bond (for example, C═C), or when atoms are bonded with a triple bond (for example, C≡C), each of the numbers of covalent bonds is calculated to be 1.

For example, when plural coordinating atoms AM1 that coordinate to M1 are present and plural coordinating atoms AM2 that coordinate to M2 are present, the number of covalent bonds present between AM1 and AM2 can be various values, and a combination of AM1 and AM2 having the value of 1 or more and 4 or less preferably exists. In addition, the fact can be rephrased such that “at least one structure in which a coordinating atom that is coordinately bonded to one transition metal atom and a coordinating atom that is coordinately bonded to another transition metal atom are bonded via 1 to 4 covalent bonds is present.”

The number of covalent bonds present between AM1 and AM2 is preferably 1 or more and 3 or less, more preferably 1 or more and 2 or less, and particularly preferably 1. As the number of covalent bonds present between AM1 and AM2 are smaller, a distance between M1 and M2 is closer.

In addition to the above, two transition metal atoms (M1 and M2) selected from plural transition metal atoms contained in the complex monomer 1 are particularly preferably bonded to the same coordinating atom, which means that M1 and M2 are crosslinkingly coordinated to the same coordinating atom. When M1 and M2 are crosslinkingly coordinated to the same coordinating atom as described above, the distance between M1 and M2 is close. When the distance between M1 and M2 is close as described above, an interaction between the two transition metal atoms is easily exhibited, and thus, catalyst activities of the complex monomer 1 and a modified polymer complex formed using the complex monomer 1 become higher.

Both of the above-described AM1 and AM2 may also be coordinating atoms in a polydentate ligand, or may be coordinating atoms in a ligand other than the polydentate ligand. A coordinating atom that crosslinkingly coordinates two transition metal atoms in the complex monomer 1 may be a coordinating atom in a polydentate ligand, or may be a coordinating atom in a ligand other than the polydentate ligand.

From the viewpoints of heat stability and the activity in the case of applying the complex monomer 1 for a catalyst, the complex monomer 1 preferably has the following structure (a) or (b), and more preferably has the structures of (a) and (b).

(a) At least one structure in which two transition metal atoms are coordinately bonded to the same coordinating atom is present.

(b) At least one structure in which a coordinating atom that is coordinately bonded to one transition metal atom and a coordinating atom that is coordinately bonded to another transition metal atom are bonded via 1 to 4 covalent bonds is present.

The structure (a) in which two transition metal atoms are coordinately bonded to the same coordinating atom is referred to as, for example, a structure in which the two transition metal atoms M¹ and M² are bonded to the same coordinating atom O¹ in the general formula (4) described later.

A polydentate ligand preferably has a structure expressed by the following general formula (2):

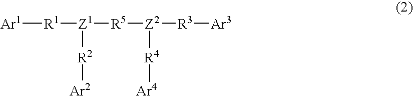

wherein Ar¹, Ar², Ar³ and Ar⁴ (hereinafter may be denoted as Ar¹ to Ar⁴) each independently denotes a nitrogen-containing aromatic heterocyclic group, R¹, R², R³, R⁴ and R⁵ (hereinafter may be denoted as R¹ to R⁵) each independently denotes a divalent group, Z¹ and Z² each independently denotes a nitrogen atom or a trivalent group, and at least one of Ar¹ to Ar⁴ and R¹ to R⁵ has a polymerizable functional group.

A part or all of coordinating atoms in the polydentate ligand expressed by the general formula (2) are preferably nitrogen atoms present on nitrogen-containing aromatic heterocyclic groups of Ar¹ to Ar⁴. The complex monomer 1 having a polydentate ligand containing such nitrogen atoms as coordinating atoms, a polymer complex formed using the complex monomer 1, and a modified product of the polymer complex are excellent in a redox catalyst activity, in particular, a catalyst activity in a hydrogen peroxide decomposition reaction.

Examples of Ar¹ to Ar⁴ in the general formula (2) include nitrogen-containing aromatic heterocyclic groups such as an imidazolyl group, a pyrazolyl group, a 2H-1,2,3-triazolyl group, a 1H-1,2,4-triazolyl group, a 4H-1,2,4-triazolyl group, a 1H-tetrazolyl group, an oxazolyl group, an isooxazolyl group, a thiazolyl group, an isothiazolyl group, a furazyl group, a pyridyl group, a pyrazyl group, a pyrimidyl group, a pyridazyl group, a 1,3,5-triazilyl group, and a 1,3,4,5-tetrazilyl group. These aromatic heterocyclic rings may be their condensed ring groups, such as a benzimidazoyl group, a 1H-indazoyl group, a benzoxazoyl group, a benzothiazoyl group, a quinolyl group, an isoquinolyl group, a cynnolyl group, a quinazoyl group, a quinoxalyl group, a phthalazyl group, a 1,8-naphthyridyl group, a pteridyl group, a carbazolyl group, a phenanthridyl group, a 1,10-phenanthrolyl group, a puryl group, a pteridyl group or a permidyl group. A condensed ring represents a cyclic structure in which each ring shares 2 or more atoms in a cyclic compound having 2 or more rings, as described in “Comprehensive Dictionary on Chemistry” (first edition, 1994, Tokyo Kagaku Dozin Co., Ltd.).

Ar¹ to Ar⁴ in the general formula (2) are preferably a benzimidazoyl group, a pyridyl group, an imidazoyl group, a pyrazoyl group, an oxazoyl group, a thiazolyl group, an isoxazolyl group, an isothiazolyl group, a pyradyl group, a pyrimidyl group, and a pyridazyl group; more preferably a benzimidazoyl group, a pyridyl group, an imidazoyl group, a pyrazoyl group, a pyradyl group, a pyrimidyl group, and a pyridazyl group; and further more preferably a benzimidazoyl group, a pyridyl group, an imidazoyl group, and a pyrazoyl group.

Ar¹ to Ar⁴ in the general formula (2) may have substituents. Substitution positions, numbers, and combinations of the substituents are arbitrary. Polymerizable functional groups described later may be bonded to the aromatic heterocyclic groups.

R⁵ in the general formula (2) is a divalent group that may have a coordinating atom or a group containing a coordinating atom, and selected from an alkylene group, a divalent aromatic group, and an organic group containing a divalent hetero atom shown below, and may be a group obtained by arbitrarily linking and combining these groups.

Examples of an alkylene group of R⁵ include alkylene groups that are obtained by removing two hydrogen atoms from a saturated hydrocarbon molecule having about 1 to 50 carbon atoms in total such as methane, ethane, propane, butane, octane, decane, icosane, triacontane, pentacontane, cycloheptane and cyclohexane. The number of carbon atoms contained in an alkylene group of R⁵ is preferably 1 to 30, more preferably 1 to 16, further more preferably 1 to 8, and particularly preferably 1 to 4. In addition, the alkylene group may be substituted with polymerizable functional groups described later.

Examples of the divalent aromatic group of R⁵ include groups that are obtained by removing two hydrogen atoms from an aromatic compound, a heterocyclic compound, or these compounds having substituents, such as benzene, naphthalene, anthracene, tetracene, biphenyl, acenaphthylene, phenalene, pyrene, furan, thiophene, pyrrole, pyridine, oxazole, isoxazole, thiazole, isothiazole, imidazole, pyrazole, pyrazine, pyrimidine, pyridazine, benzofuran, isobenzofuran, 1-benzothiophene, 2-benzothiophene, indole, isoindole, indolizine, carbazole, xanthene, quinoline, isoquinoline, 4H-quinolizine, phenanthridine, acrydine, 1,8-naphthyridine, benzimidazole, 1H-indazole, quinoxaline, quinazoline, cinnoline, phthalazine, purine, pteridine, perimidine, 1,10-phenanthroline, thianthorene, phenoxathiin, phenoxadine, phenothiazine, phenazine and phenarsazine.

Among them, as a divalent aromatic group as R⁵, preferable are groups that are obtained by removing two hydrogen atoms from a compound selected from benzene, phenol, p-cresol, naphthalene, biphenyl, furan, thiophene, pyrrole, pyridine, oxazole, isoxazole, thiazole, isothiazole, imidazole, pyrazole, pyrazine, pyrimidine, pyridazine, benzofuran, isobenzofuran, 1-benzothiophene, 2-benzothiophene, indole, isoindole, indolizine, carbazole, xanthene, quinoline, isoquinoline, 1,8-naphthyridine, benzimidazole, 1H-indazole, quinoxaline, quinazoline, cinnoline, phthalazine, purine, pteridine and perimidine; more preferable are groups that are obtained by removing two hydrogen atoms from a compound selected from benzene, naphthalene, biphenyl, pyrrole, pyridine, oxazole, isoxazole, thiazole, isothiazole, imidazole, pyrazole, pyrazine, pyrimidine, pyridazine, indole, isoindole, quinoline, isoquinoline, 1,8-naphthyridine, benzimidazole, 1H-indazole, quinoxaline, quinazoline, cinnoline and phthalazine; further more preferable are groups that are obtained by removing two hydrogen atoms from a compound selected from benzene, phenol, p-cresol, naphthalene, biphenyl, pyrrole, pyridine, imidazole, pyrazole, pyrazine, pyridazine, indole, isoindole, quinoline, isoquinoline, 1,8-naphthyridine, benzimidazole, 1H-indazole, quinoxaline, quinazoline, cinnoline and phthalazine; and particularly preferable are groups that are obtained by removing two hydrogen atoms from a compound selected from phenol, p-cresol, pyridine, pyrazole, pyridazine, 1,8-naphthyridine, 1H-indazole and phthalazine. In addition, a divalent aromatic group as R⁵ may be substituted with polymerizable functional groups described later.

When R⁵ in the general formula (2) is a divalent group containing a hetero atom, groups expressed by the following formulas (E-1) to (E-10) or groups containing these groups can be mentioned as examples of R⁵.

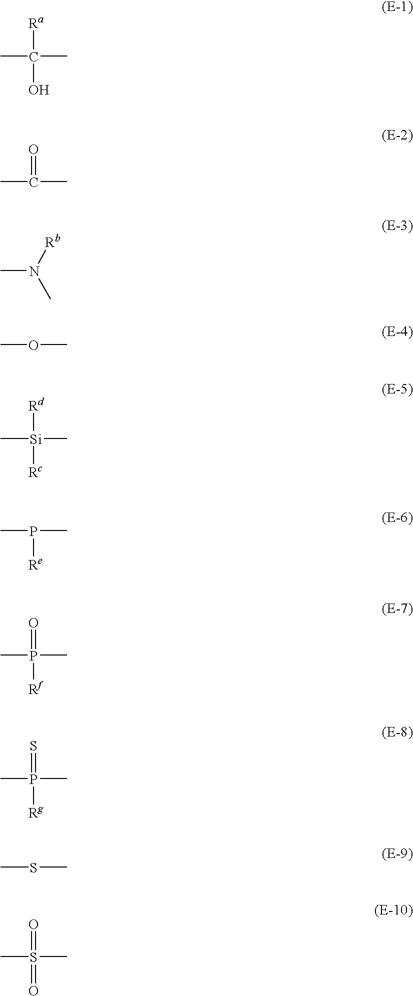

In the formulas (E-1) to (E-10), R^{a}, R^{e}, R^{f} and R^{g} denote an alkyl group having 1 to 50 carbon atoms, an aromatic group having 2 to 60 carbon atoms, an alkoxy group having 1 to 50 carbon atoms, an aryloxy group having 2 to 60 carbon atoms, a hydroxyl group, or a hydrogen atom; R^{b} denotes an alkyl group having 1 to 50 carbon atoms, an aromatic group having 2 to 60 carbon atoms, or a hydrogen atom; and R^{d} and R^{c} each denote an alkyl group having 1 to 50 carbon atoms, or an aromatic group having 2 to 60 carbon atoms.

As a divalent group containing a hetero atom as R⁵ in the general formula (2), (E-1), (E-2), (E-3), (E-4), (E-5), (E-7), (E-8) and (E-10) are preferable, (E-1), (E-2), (E-4), (E-7) and (E-10) are more preferable, and (E-1) and (E-7) are further more preferable.

R⁵ in the general formula (2) preferably contains a functional group capable of coordinating to a transition metal atom. Examples of the functional group capable of coordinating to a transition metal atom include a hydroxyl group, a carboxyl group, a mercapto group, a sulfonic acid group, a phosphonic acid group, a nitro group, a cyano group, an ether group, an acyl group, an ester group, an amino group, a carbamoyl group, an acid amide group, a phospholyl group, a thiophospholyl group, a sulfide group, a sulfonyl group, a pyrrolyl group, a pyridyl group, an oxazolyl group, an isoxazolyl group, a thiazolyl group, an isothizoyl group, an imidazolyl group, a pyrazolyl group, a pyrazyl group, a pyrimidyl group, a pyridazyl group, an indolyl group, an isoindolyl group, a carbazolyl group, a quinolyl group, an isoqunolyl group, a 1,8-naphthyridyl group, a benzimidazolyl group, a 1H-indazolyl group, a quinoxalyl group, a quinazolyl group, a cinnolyl group, a phtalazyl group, a puryl group, a pteridyl group and a permidyl group. Among these examples, preferable are a hydroxyl group, a carboxyl group, a sulfonic acid group, a phosphonic acid group, a nitro group, a cyano group, an ether group, an acyl group, an amino group, a phospholyl group, a thiophospholyl group, a sulfonyl group, a pyrrolyl group, a pyridyl group, an oxazolyl group, an isoxazolyl group, a thiazolyl group, an isothizoyl group, an imidazolyl group, a pyrazolyl group, a pyrazyl group, a pyrimidyl group, a pyridazyl group, an indolyl group, an isoindolyl group, a quinolyl group, an isoqunolyl group, a 1,8-naphthyridyl group, a benzoimidazolyl group, a 1H-indazolyl group, a quinoxalyl group, a quinazolyl group, a cinnolyl group, a phtalazyl group, a puryl group, a pteridyl group and a permidyl group; and more preferable are a hydroxyl group, a carboxyl group, a sulfonic acid group, a phosphonic acid group, a cyano group, an ether group, an acyl group, an amino group, a phospholyl group, a sulfonyl group, a pyridyl group, an imidazolyl group, a pyrazolyl group, a pyrimidyl group, a pyridazyl group, a quinolyl group, an isoqunolyl group, a 1,8-naphthyridyl group, a benzimidazolyl group, a 1H-indazolyl group, a cinnolyl group, a phtalazyl group and a pteridyl group.

As R⁵ in the general formula (2), a group expressed by the following chemical formula (R5-1), (R5-2), (R5-3) or (R5-4) is preferable, and a group expressed by the following chemical formula (R5-1) is more preferable.

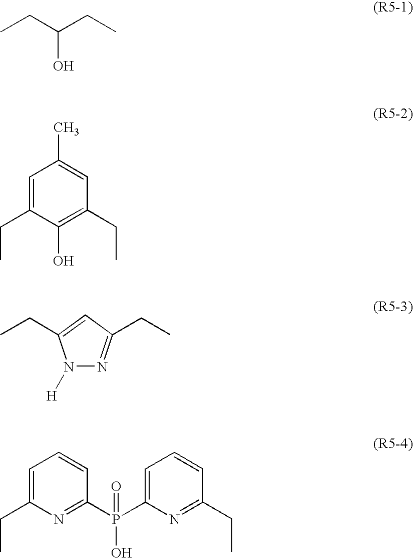

A hydroxyl group in the chemical formulas (R5-1) and (R5-2), a pyrazole ring in (R5-3), and a phosphinic acid group in (R5-4) sometimes become anionic by releasing a proton in coordinating to a metal atom as ligands.

In the general formula (2), R¹ to R⁴ are divalent groups that may be substituted, and may be the same or different from one another. R¹ to R⁴ may be an alkylene group, a divalent aromatic group or a divalent group containing a hetero atom, or a divalent group obtained by arbitrarily linking and combining these groups being the same as R⁵, and a methylene group, a 1,1-ethylene group, a 2,2-propylne group, a 1,2-ethylene group and a 1,2-phenylene group are preferable, and a methylene group and a 1,2-ethylene group are more preferable.

Z¹ and Z² in the general formula (2) are selected from a nitrogen atom or a trivalent group, and examples of the trivalent group include groups expressed by any of the following general formulas (Z-1), (Z-2), (Z-3), (Z-4), (Z-5), (Z-6), and (Z-7). Either Z¹ or Z² is preferably a nitrogen atom, and it is more preferable that both are nitrogen atoms.

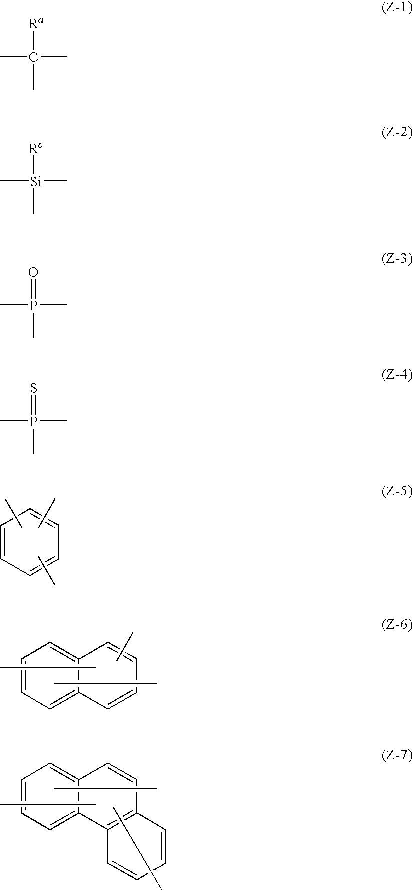

R^{a} in the general formulas (Z-1) and (Z-2) denotes an alkyl group having 1 to 50 carbon atoms, an aromatic group having 2 to 60 carbon atoms, an alkoxy group having 1 to 50 carbon atoms, an aryloxy group having 2 to 60 carbon atoms, a hydroxyl group, or a hydrogen atom, and R^{c} denotes an alkyl group having 1 to 50 carbon atoms, or an aromatic group having 2 to 60 carbon atoms.

At least one of Ar¹ to Ar⁴ and R¹ to R⁵ in the general formula (2) has a polymerizable functional group, and examples of the polymerizable functional group include a vinyl group, an acrylic group, a methacrylic group, an allyl group, a propenyl group, a butenyl group, a butadinyl group, a styryl group, a 2-vinylbenzyl group, a 3-vinylbenzyl group, and a 4-vinylbenzyl group; a vinyl group, a styryl group, an allyl group, a 2-vinylbenzyl group, a 3-vinylbenzyl group, and a 4-vinylbenzyl group are preferable; and a vinyl group, a styryl group, an allyl group and a 4-vinylbenzyl group are more preferable.

Among the polydentate ligands expressed by the general formula (2), a polydentate ligand having a structure expressed by the following general formula (3a) or (3b) is preferable.

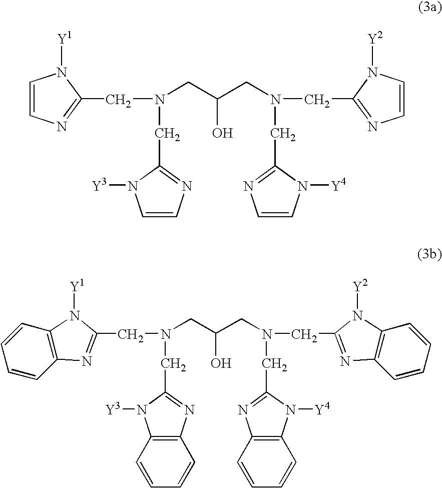

In the general formula (3a) or (3b), each of Y¹, Y², Y³ and Y⁴ independently denotes a hydrogen atom, an alkyl group having 1 to 50 carbon atoms, or an aromatic group having 2 to 60 carbon atoms, and at least one of Y¹, Y², Y³ and Y⁴ is an alkyl group having 1 to 50 carbon atoms which has a polymerizable functional group, or an aromatic group having 2 to 60 carbon atoms which has a polymerizable functional group.

Similarly to the general formula (2), a hydroxyl group in the general formula (3a) or (3b) sometimes becomes anionic by releasing a proton in coordinating to a transition metal atom as a ligand. Examples of a polymerizable functional group in Y¹ to Y⁴ include a vinyl group, an acrylic group, a methacrylic group, an allyl group, a propenyl group, a butenyl group, a butadinyl group, a styryl group, a 2-vinylbenzyl group, a 3-vinylbenzyl group, and a 4-vinylbenzyl group; a vinyl group, a styryl group, an allyl group, a 2-vinylbenzyl group, a 3-vinylbenzyl group, and a 4-vinylbenzyl group are preferable; and a vinyl group, a styryl group, an allyl group, and a 4-vinylbenzyl group are more preferable.

The complex monomer 1 may have another ligand in addition to the above-described polydentate ligand. Such another ligand may be an ionic compound or an electrically neutral compound, and when the complex monomer 1 has plural other ligands, these other ligands may be the same or different from one another.

Examples of an electrically neutral compound in the other ligands except for the polydentate ligand include: nitrogen atom-containing compounds such as ammonia, pyridine, pyrrole, pyridazine, pyrimidine, pyrazine, 1,2,4-triazine, pyrazole, imidazole, 1,2,3-triazole, oxazole, isoxazole, 1,3,4-oxadiazole, thiazole, isothiazole, indole, indazole, quinoline, isoquinoline, phenanthridine, cinnoline, phthalazine, quinazoline, quinoxaline, 1,8-naphthylidine, acridine, 2,2′-bipyridine, 4,4′-bipyridine, 1,10-phenanthroline, ethylenediamine, propylenediamine, phenylenediamine, cyclohexanediamine, pyridine N-oxide, 2,2′-bipyridine N,N′-dioxide, oxamide, dimethyl glyoxime and o-aminophenol; oxygen-containing compounds such as water, phenol, oxalic acid, catechol, salicylic acid, phthalic acid, 2,4-pentanedione, 1,1,1-trifluoro-2,4-pentanedione, hexafluoropentanedione, 1,3-diphenyl-1,3-propanedione, and 2,2′-binaphthol; sulfur-containing compounds such as dimethyl sulfoxide and urea; and phosphorous-containing compounds such as 1,2-bis(dimethylphosphino)ethane and 1,2-phenylenebis(dimethylphosphine). Among the above-described ligands that are electrically neutral compounds, preferable are ammonia, pyridine, pyrrole, pyridazine, pyrimidine, pyrazine, 1,2,4-triazine, pyrazole, imidazole, 1,2,3-triazole, oxazole, isoxazole, 1,3,4-oxadiazole, indole, indazole, quinoline, isoquinoline, phenanthridine, cinnoline, phthalazine, quinazoline, quinoxaline, 1,8-naphthylidine, acridine, 2,2′-bipyridine, 4,4′-bipyridine, 1,10-phenanthroline, ethylenediamine, propylenediamine, phenylenediamine, cyclohexanediamine, pyridine N-oxide, 2,2′-bipyridine N,N′-dioxide, oxamide, dimethyl glyoxime, o-aminophenol, water, phenol, oxalic acid, catechol, salicylic acid, phthalic acid, 2,4-pentanedione, 1,1,1-trifluoro-2,4-pentanedione, hexafluoropentanedione, 1,3-diphenyl-1,3-propanedione and 2,2′-binaphthol; more preferable are ammonia, pyridine, pyrrole, pyridazine, pyrimidine, pyrazine, 1,2,4-triazine, pyrazole, imidazole, 1,2,3-triazole, oxazole, isoxazole, 1,3,4-oxadiazole, indole, indazole, quinoline, isoquinoline, phenanthridine, cinnoline, phthalazine, quinazoline, quinoxaline, 1,8-naphthylidine, acridine, 2,2′-bipyridine, 4,4′v-bipyridine, 1,10-phenanthroline, ethylenediamine, propylenediamine, phenylenediamine, cyclohexanediamine, pyridine N-oxide, 2,2′-bipyridine N,N′-dioxide, o-aminophenol, phenol, catechol, salicylic acid, phthalic acid, 1,3-diphenyl-1,3-propanedione, and 2,2′-binaphthol; and further preferable are pyridine, pyrrole, pyridazine, pyrimidine, pyrazine, pyrazole, imidazole, oxazole, indole, quinoline, isoquinoline, acridine, 2,2′-bipyridine, 4,4′-bipyridine, 1,10-phenanthroline, phenylenediamine, pyridine N-oxide, 2,2′-bipyridine N,N′-dioxide, o-aminophenol and phenol.

Examples of an anionic ligand among other ligands except for the polydentate ligand include a hydroxide ion, peroxide, super oxide, a cyanide ion, a thiocyanate ion, halide ions such as a fluoride ion, a chloride ion, a bromide ion and an iodide ion, a sulfate ion, a nitrate ion, a carbonate ion, a perchlorate ion, tetraarylborate ions such as a tetrafluoroborate ion and a tetraphenylborate ion, a hexafluorophosphate ion, sulfonate ions such as a methanesulfonate ion, a trifluoromethanesulfonate ion, a p-toluenesulfonate ion, a benzenesulfonate ion and a dodecylbenzenesulfonate ion, a dodecylsulfate ion, a sulfuric acid ester ion, a phosphate ion, a phosphite ion, a phenylphosphonate ion, a diphenylphosphonate ion, an acetate ion, a trifluoroacetate ion, a propionate ion, a benzoate ion, a hydroxyl ion, a metal oxide ion, a methoxide ion, an ethoxide ion, a vinylbenzoate ion, an acrylate ion, a methacrylate ion, and an anionic ligand having a structure expressed by the following general formula (40):

G⁰¹—(OCH₂CH(R))ₙOR (40)

wherein R denotes a hydrogen atom, an alkyl group having 1 to 50 carbon atoms which may have a substituent, or an aryl group having 6 to 50 carbon atoms which may have a substituent, and G⁰¹ denotes a substituent containing a functional group which has a structure expressed by any of the following formulas (4-1), (4-2), (4-3) and (4-4), respectively. In the formula (4-4), R has the same meaning as the above.

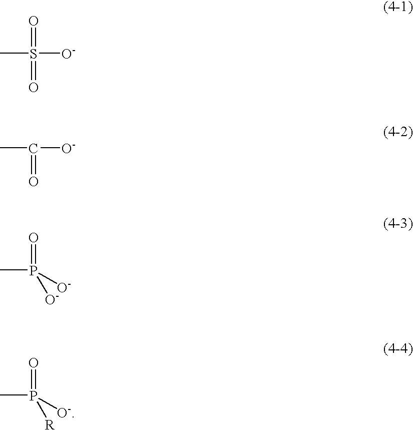

Among the above-described anionic ligands, preferable are a hydroxide ion, a sulfate ion, a nitrate ion, a carbonate ion, a perchlorate ion, a tetrafluoroborate ion, a tetraphenylborate ion, a hexafluorophosphate ion, a methanesulfonate ion, a trifluoromethanesulfonate ion, a p-toluenesulfonate ion, a benzenesulfonate ion, a sulfuric acid ester ion, a phosphate ion, a phosphite ion, a phenylphosphonate ion, a diphenylphosphonate ion, an acetate ion, a trifluoroacetate ion, a vinyl benzoate ion, an acrylate ion, a methacrylate ion, and anionic ligands expressed by the general formula (40) which has G⁰¹ expressed by (G-1), (G-2), (G-3), (G-4), (G-5), (G-6), (G-7), (G-8), (G-9), (G-10), (G-11), or (G-12) in the general formula (40). Among them, more preferable are a hydroxide ion, a sulfate ion, a nitrate ion, a carbonate ion, a tetraphenylborate ion, a trifluoromethanesulfonate ion, a p-toluenesulfonate ion, a sulfuric acid ester ion, an acetate ion, a trifluoroacetate ion, a vinylbenzoate ion, an acrylate ion, a methacrylate ion, and anionic ligands expressed by the general formula (40) which has G⁰¹ expressed by (G-1), (G-2), (G-3), (G-4), (G-5), (G-6), (G-7), (G-8), (G-9), (G-10), (G-11), or (G-12) in the general formula (40).

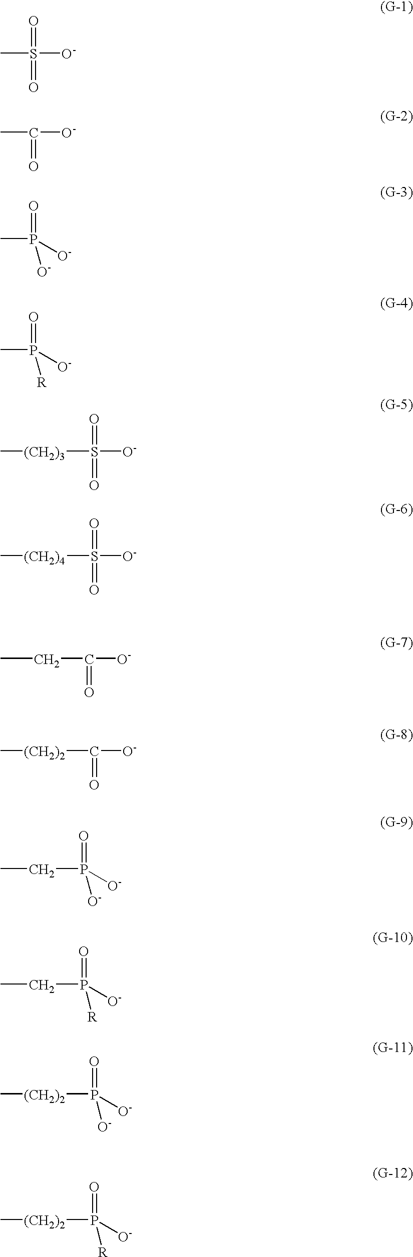

Further, the ions shown as the anionic ligand may act as a counter ion to electrically neutralize the complex monomer 1 itself. Additionally, using various counter ions suitably can also adjust solubility or dispersibility of the complex monomer 1 (polynuclear complex) in a solvent.

Furthermore, the complex monomer 1 may have a counter ion with a cationic property to maintain electrical neutrality. Examples of the cationic counter ion include alkali metal ions, alkaline earth metal ions, tetraalkylammonium ions such as a tetra(n-butyl)ammonium ion and a tetraethylammonium ion, and tetraarylphosphonium ions such as a tetraphenylphosphonium ion, and specific examples include a lithium ion, a sodium ion, a potassium ion, a rubidium ion, a cesium ion, a magnesium ion, a calcium ion, a strontium ion, a barium ion, a tetra(n-butyl)ammonium ion, a tetraethylammonium ion and a tetraphenylphosphonium ion; and more preferable are a tetra(n-butyl)ammonium ion, a tetraethylammonium ion and a tetraphenylphosphonium ion. Among these ions, a tetra(n-butyl)ammonium ion and a tetraethylammonium ion are preferable as a cationic counter ion.

Next, the complex monomer of the second embodiment will be specifically described. Hereinafter, the complex monomer of the second embodiment may be simply referred to as the complex monomer 2.

The complex monomer 2 meets the following conditions (i′) to (iv′):
(i′) the complex monomer has one or more transition metal atoms;
(ii′) the complex monomer has a polydentate ligand containing three or more coordinating atoms that are coordinately bonded to the transition metal atoms;
(iii′) the polydentate ligand has one or more polymerizable functional groups; and
(iv′) the complex monomer has any structure of an organic acid salt structure, an amine salt structure, an ammonium salt structure, a pyridinium salt structure, an imidazolium salt structure, a hydroxyl group structure, an ether structure and an acid amide structure.

The complex monomer 2 has one or more transition metal atoms. As a result, a catalyst activity to decompose hydrogen peroxide into water and oxygen while suppressing generation of free radicals (hydroxyl radical, hydroperoxy radical, and the like) can be given to the complex monomer 2 and a polymer complex obtained by polymerization or copolymerization of the complex monomer 2. The transition metal atoms that the complex monomer 2 has may be uncharged or charged ions. When plural transition metal atoms are contained in the complex monomer 2, the plural transition metal atoms may be the same or different.

The transition metal atoms in the complex monomer 2 are the same as the transition metal atoms in the above-described complex monomer 1.

At least one of the ligands contained in the complex monomer 2 is a polydentate ligand. When the ligand is a polydentate ligand, the more stable complex monomer 2 can be formed due to a chelate effect of the polydentate ligand. The number of coordinating atoms coordinately bonding to a transition metal atom in a polydentate ligand is 3 or more, preferably 3 or more and 50 or less, further more preferably 4 or more and 25 or less, and particularly preferably 6 or more and 20 or less.

A polydentate ligand in the complex monomer 2 has one or more polymerizable functional groups. The complex monomer 2 is polymerized or the complex monomer 2 and a comonomer are copolymerized to form into a high molecular compound with the use of polymerization reactivity of the polymerizable functional groups, thereby making it possible to easily derive to a nonuniform complex catalyst.

The number of polymerizable functional groups contained in the complex monomer 2 is preferably 1 or more and 20 or less, more preferably 2 or more and 10 or less, and particularly preferably 4 or more and 8 or less, from the viewpoint of synthesis.

Examples of the polymerizable functional groups include a vinyl group, an acrylic group, a methacrylic group, an allyl group, a propenyl group, a butenyl group, a butadinyl group, a styryl group, a 2-vinylbenzyl group, a 3-vinylbenzyl group, and a 4-vinylbenzyl group; a vinyl group, a styryl group, an allyl group, a 2-vinylbenzyl group, a 3-vinylbenzyl group, and a 4-vinylbenzyl group are preferable; and a vinyl group, styryl group, an allyl group, and a 4-vinylbenzyl group are more preferable.

The complex monomer 2 has any of structures from an organic acid salt structure, an amine salt structure, an ammonium salt structure, a pyridinium salt structure, an imidazolium salt structure, a hydroxyl group structure, an ether structure and an acid amide structure. These structures preferably have any of functional groups expressed by the following general formulas (1-1), (1-2), (1-3), (1-4), (1-5), (1-6), (1-7), (1-8), and (1-9). The complex monomer 2 has such a hydrophilic functional group in combination, which makes it possible to disperse the complex monomer 2 in a polar solvent, and a particulate polymer complex can be obtained by performing polymerization of the complex monomer 2 in such a reaction system. Among the following general formulas (1-1) to (1-9), as a functional group that the complex monomer 2 has, functional groups expressed by the general formulas (1-1), (1-2), (1-3), (1-4), (1-5), and (1-6) are preferable, functional groups expressed by the general formulas (1-1), (1-3), (1-4), and (1-5) are more preferable, and a functional group expressed by the general formula (1-1) is particularly preferable.

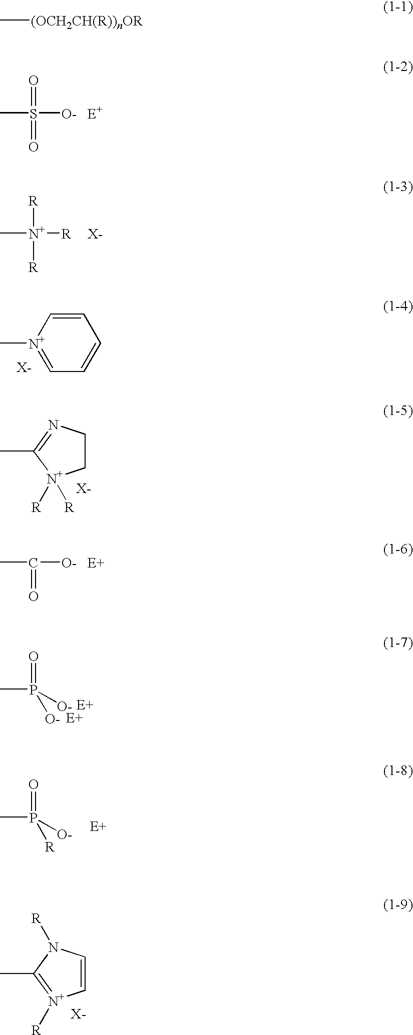

In the general formulas (1-1), (1-2), (1-3), (1-4), (1-5), (1-6), (1-7), (1-8) and (1-9), n denotes an integer of 1 or more and 500 or less. E⁺ denotes a proton, a lithium ion, a sodium ion, a potassium ion, a rubidium ion, a cesium ion or an ammonium ion. R denotes a hydrogen atom, an alkyl group having 1 to 50 carbon atoms which may have a substituent, or an aryl group having 6 to 50 carbon atoms which may have a substituent. X⁻ denotes a fluoride ion, a chloride ion, a bromide ion, an iodide ion, a methane sulfonate ion, or a trifluoromethane sulfonate ion.

Such a complex monomer 2 shows good dispersibility in a solvent such as water, a particle dispersing radical polymerization method such as emulsion polymerization may be applied thereto, and the polymerization method can induce a particulate polymer complex. Further, the obtained particulate polymer complex has a large surface area, and is optimal for use as a catalyst. The complex monomer 2 and a polymer complex can have plural metal atoms as central atoms and can be applied to a nonuniform redox catalyst.

The complex monomer 2 preferably has a structure expressed by the following general formula (2-1):

(L⁰¹)ₚ(M)ₘ(L⁰²)_{q} (2-1)

wherein m denotes an integer of 1 or more and 20 or less, p denotes an integer of 1 or more and 5 or less, and q denotes an integer of 1 or more and 20 or less. M denotes a transition metal atom, and when there are two or more transition metal atoms in the structure, they may be the same elements or may be different elements from one another. L⁰¹ denotes a polydentate ligand having a substituent containing a polymerizable functional group or a functional group expressed by the general formula (1-1) and having 3 or more coordinating atoms including a nitrogen coordinating atom, and when there are two or more polydentate ligands of L⁰¹, they may be the same polydentate ligands or may be different polydentate ligands from one another. L⁰² denotes a ligand or a counter ion, which has a substituent containing a polymerizable functional group or a functional group expressed by the general formula (1-1), and when there are two or more ligands or counter ions of L⁰², they may be the same ligands or counter ions, or may be different ligands or counter ions from one another. Here, a combination of the substituents in L⁰¹ and L⁰² is a combination having both of a polymerizable functional group and a functional group expressed by the general formula (1-1).

That is, in the general formula (2-1), L⁰¹ and L⁰² each have either of a polymerizable functional group or a substituent containing a functional group expressed by the general formula (1-1), and a combination of these substituents is a combination in which the complex monomer 2 expressed by the general formula (2-1) has both of a polymerizable functional group and a substituent containing a functional group expressed by the general formula (1-1). In particular, a combination in which L⁰¹ has a polymerizable functional group and L⁰² has a substituent containing a functional group expressed by the general formula (1-1) is preferable. Having such a combination facilitates synthesis of a ligand raw material and the complex monomer 2. The complex monomer 2 has a functional group expressed by the general formula (1-1), thereby being excellent in a dispersion ability and/or an emulsification ability in a polar solvent, particularly, a solvent containing water. Therefore, for example, when the complex monomer 2 and a comonomer are copolymerized to form a polymer complex, an excellent emulsion polymerization property is exerted.

From the viewpoints of an activity in the case of application for a catalyst, the complex monomer 2 preferably has the following structure (a′) or (b′), and more preferably has the structures (a′) and (b′).

(a′) At least one structure having two or more transition metal atoms, in which two transition metal atoms among the two or more transition metal atoms are coordinately bonded to the same coordinating atom, is present.

(b′) At least one structure having two or more transition metal atoms, in which a coordinating atom that is coordinately bonded to one transition metal atom among the two or more transition metal atoms and a coordinating atom that is coordinately bonded to another transition metal atom other than the one transition metal atom among the two or more transition metal atoms are bonded via 1 to 4 covalent bonds, is present.

The structure (a′) in which two transition metal atoms are coordinately bonded to the same coordinating atom is referred to as, for example, a structure in which the two transition metal atoms M¹ and M² are bonded to the same coordinating atom O¹ in the general formula (4) described later.

The total number of polymerizable functional groups that L⁰¹ or L⁰² in the general formula (2-1) has is preferably 1 or more and 20 or less, more preferably 2 or more and 10 or less, and particularly preferably 4 or more and 8 or less, from the viewpoint of synthesis. Examples of the polymerizable functional groups that L⁰¹ or L⁰² has include a vinyl group, an acrylic group, a methacrylic group, an allyl group, a propenyl group, a butenyl group, a butadinyl group, a styryl group, a 2-vinylbenzyl group, a 3-vinylbenzyl group, and a 4-vinylbenzyl group; a vinyl group, a styryl group, an allyl group, a 2-vinylbenzyl group, a 3-vinylbenzyl group, and a 4-vinylbenzyl group are preferable; and a vinyl group, a styryl group, an allyl group, and a 4-vinylbenzyl group are more preferable.

The number m of transition metal atoms contained in the complex monomer 2 expressed by the general formula (2-1) is preferably 2 or more and 8 or less, more preferably 2 or more and 4 or less, and particularly preferably 2 or 3. Setting the number m of transition metal atoms to the above-described preferable value makes it possible to surely impart a redox catalyst ability capable of multielectron transfer to the complex monomer 2 and a polymer complex obtained from the complex monomer 2, and at the same time, makes synthesis of the complex monomer 2 and the polymer complex obtained from the complex monomer 2 easy.

L⁰¹ in the general formula (2-1) contains a nitrogen coordinating atom, thereby making it possible to further improve a redox catalyst ability of the complex monomer 2 and the polymer complex obtained from the complex monomer 2.

The number of coordinating atoms in L⁰¹ in the general formula (2-1) is preferably 3 or more and 50 or less, more preferably 4 or more and 25 or less, and particularly preferably 6 or more and 20 or less. The number of substituents containing a functional group expressed by the general formula (1-1) in L⁰¹ is preferably 1 or more and 10 or less, more preferably 1 or more and 6 or less, and particularly preferably 1 or more and 4 or less.

The number of substituents containing a functional group expressed by the general formula (1-1) in L⁰² is preferably 1 or more and 10 or less, more preferably 1 or more and 4 or less, and particularly preferably 1 or more and 2 or less. One example of L⁰² having a substituent containing a functional group expressed by the general formula (1-1) is one expressed by the general formula (40) described above.

The number of polymerizable functional groups contained in L⁰² is preferably 1 or more and 6 or less, more preferably 1 or more and 4 or less, and particularly preferably 1. Examples of L⁰² having a polymerizable functional group include acrylate, methacrylate, vinylpyridine, vinylimidazole, isopropenyloxazoline, acrylonitrile, methacrylonitrile, acryloamide, methacrylamide, vinylpyrrolidone, vinylaniline, vinylanilide, styrenesulfonate, vinylsulfonate, vinylphosphonate, and vinylbenzoate; preferable are acrylate, methacrylate, vinylpyridine, vinylimidazole, isopropenyloxazoline, styrenesulfonate, vinylsulfonate, vinylphosphonate, and vinylbenzoate; more preferable are acrylate, methacrylate, vinylpyridine, vinylimidazole, isopropenyloxazoline, vinylphosphonate, and vinylbenzoate; and particularly preferable are acrylate, methacrylate, vinylphosphonate, and vinylbenzoate.

The complex monomer 2 shown by the general formula (2-1) may further have another ligand that is neither L⁰¹ nor L⁰² in combination. The other ligand may be an ionic or electrically neutral compound, and when the complex monomer 2 has the plural ligands, these ligands may be the same or different from one another.

The above-described other ligands that are neither L⁰¹ nor L⁰² in the complex monomer 2 are the same as the other ligands besides a polydentate ligand in the complex monomer 1 described above.

The complex monomer 2 preferably has two or more transition metal atoms. Moreover, from the viewpoint that the complex monomer 2 and a polymer complex obtained from the complex monomer 2 are used as redox catalysts, in particular, at least two transition metal atoms among plural transition metal atoms are closely located in a molecule.

When two transition metal atoms in the complex monomer 2 are assumed to be M1 and M2, one of coordinating atoms that coordinates to M1 is assumed to be AM1, and one of coordinating atoms that coordinates to M2 is assumed to be AM2, the number of covalent bonds present between AM1 and AM2 can be calculated and used as “an index of closely located transition metal atoms.” Herein, when AM1 and AM2 are directly bonded, the number of covalent bonds is 1, when AM1 and AM2 are covalently bonded on the whole via one atom, the number of covalent bonds is 2, and when AM1 and AM2 are covalently bonded on the whole via n atoms, the number of covalent bonds is (n+1). When atoms are directly bonded with a double bond (for example, C═C), or when atoms are bonded with a triple bond (for example, C≡C), each of the numbers of covalent bonds is calculated to be 1.

For example, when plural coordinating atoms AM1 that coordinate to M1 are present and plural coordinating atoms AM2 that coordinate to M2 are present, the number of covalent bonds present between AM1 and AM2 can be various values, and a combination of AM1 and AM2 having the value of 1 or more and 4 or less is preferably present. Herein, the fact can be rephrased such that “at least one structure in which a coordinating atom that is coordinately bonded to one transition metal atom and a coordinating atom that is coordinately bonded to another transition metal atom other than the one transition metal atom are bonded via 1 to 4 covalent bonds is present.”

The number of covalent bonds present between AM1 and AM2 is preferably 1 or more and 3 or less, more preferably 1 or more and 2 or less, and particularly preferably 1. As the number of covalent bonds present between AM1 and AM2 is smaller, a distance between M1 and M2 is closer. As a result, an interaction between the two transition metal atoms is easily exhibited, and catalyst activities of the complex monomer 2 and a polymer complex formed using the complex monomer 2 become higher.

In addition to the above, two transition metal atoms (M1 and M2) selected from plural transition metal atoms contained in the complex monomer 2 are particularly preferably bonded to the same coordinating atom, which means that M1 and M2 are crosslinkingly coordinated to the same coordinating atom. When M1 and M2 are crosslinkingly coordinated to the same coordinating atom as described above, the distance between M1 and M2 is close. When the distance between M1 and M2 is close as described above, an interaction between the two transition metal atoms is easily exhibited, and thus, catalyst activities of the complex monomer 2 and a polymer complex formed using the complex monomer 2 become higher.

Both of the above-described AM1 and AM2 may be coordinating atoms in a polydentate ligand, or may be coordinating atoms in a ligand other than the polydentate ligand. In the complex monomer 2, a coordinating atom that crosslinkingly coordinates two transition metal atoms may be a coordinating atom in a polydentate ligand, or may be a coordinating atom in a ligand other than the polydentate ligand.

In the complex monomer 2, the polydentate ligand L⁰¹ in the general formula (2-1) preferably has a structure expressed by the general formula (2) in the above-described complex monomer 1, and more preferably has a structure expressed by the general formula (3a) or (3b).

In the complex monomer 2, the polydentate ligand L⁰² in the general formula (2-1) preferably has a structure expressed by the general formula (40) in the above-described complex monomer 1. A compound expressed by the general formula (40) in the complex monomer 2 is a compound capable of functioning as a counter ion in a catalyst, and the complex monomer 2 having such a structure is excellent in a dispersion ability and/or an emulsification ability in a polar solvent, particularly, a solvent containing water. Therefore, for example, when the complex monomer 2 and a comonomer are copolymerized to form a polymer complex, an excellent emulsion polymerization property is exerted.

In the present specification, when simply mentioning a “complex monomer,” it means the complex monomer of the first embodiment (complex monomer 1) or the complex monomer of the second embodiment (complex monomer 2).

Specific preferable examples of the complex monomer according to the present embodiment include a complex monomer having a structure expressed by the following general formula (4):

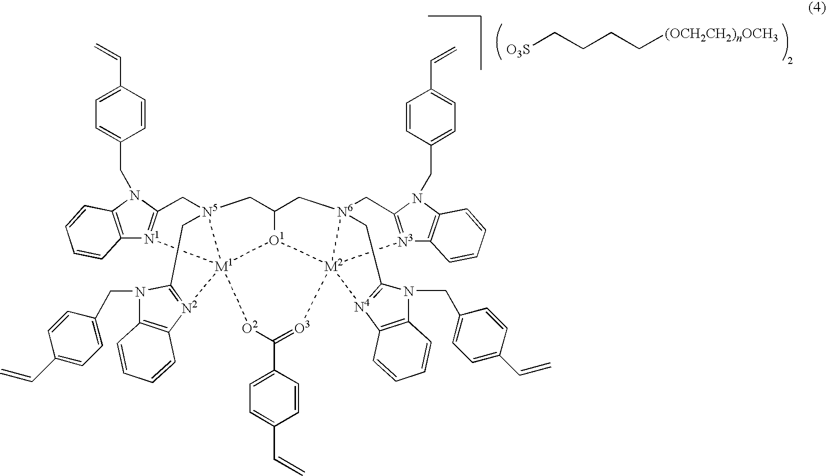

In the complex monomer expressed by the general formula (4), a polydentate ligand having 3 or more coordinating atoms has four benzimidazolyl groups as aromatic heterocyclic groups containing nitrogen coordinating atoms (Ar¹ to Ar⁴ in the general formula (2)). One nitrogen atom in the benzimidazolyl group coordinates to M¹ or M² as a coordinating atom (denoted as N¹, N², N³ or N⁴) (dotted lines connected to M¹ or M² show coordination bonds), and a 4-vinylbenzyl group having polymerization reactivity is bonded to the other nitrogen atom in this benzimidazolyl group. Groups denoted as R¹ to R⁴ in the general formula (2) are methylene groups in the general formula (4), and R⁵ in the general formula (2) is a group having a propylene group with a crosslinkable coordinating atom (denoted as O¹) in the general formula (4). Further, a p-vinyl benzoate ion is contained (having O² and O³ as coordinating atoms) as a ligand other than the above-described polydentate ligand, and two molecules of anions expressed by the chemical formula, O₃SCH₂CH₂CH₂CH₂(OCH₂CH₂)ₙOCH₃ are contained as counter ions. n in the chemical formula is about 45. In the general formula (4), the numbers expressed beside nitrogen coordinating atoms and oxygen coordinating atoms are expressed for distinction in describing the number of covalent bonds between coordinating atoms described below.

Herein, the number of covalent bonds present among coordinating atoms that coordinate to M¹ and M² respectively in a complex monomer expressed by the general formula (4) will be described. In the complex of the general formula (4), between M¹-O¹-M², M¹ and M² are (crosslinkingly) coordinated to the same coordinating atom O¹; between M¹-O²—O³-M², the minimum of the number of covalent bonds linking coordinating atoms is 2; between M¹-O¹—N⁶-M², and between M²-O¹—N⁵-M¹, the minimum of the number of covalent bonds linking coordinating atoms is 3; and between M¹-N⁵—N⁶-M², the minimum of the number of covalent bonds linking coordinating atoms is 4. That is, it can be recognized that “at least one structure in which a coordinating atom that is coordinately bonded to one transition metal atom and a coordinating atom that is coordinately bonded to another transition metal atom are bonded via 1 to 4 covalent bonds is present.”

A polynuclear complex having such a combination of coordinating atoms is a polynuclear complex having a coordination structure in which M¹ and M² are closely present, and such a polynuclear complex has a high catalyst activity and thus is preferable as a complex monomer.

### (Comonomer)

A complex monomer and a comonomer are copolymerized and thus a polymer complex can be obtained.

As the comonomer, various compounds having carbon-carbon double bonds can be used in combination with various amount ratios, but in the present embodiment, a comonomer having a structure expressed by the following general formula (1) is used.

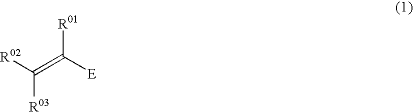

In the general formula (1), E denotes a cyano group, a carboxyl group, a formyl group, a carbamoyl group, a phosphonic acid group, a sulfonic acid group, a halogeno group, a —CONHCH₂OR⁰⁴ group or a —Si(OR⁰⁵)₃ group, each of R⁰¹, R⁰² and R⁰³ independently denotes a hydrogen atom, a halogeno group, a cyano group, a —COOR⁰⁴ group, an alkyl group having 1 to 10 carbon atoms which may have a substituent, or an aryl group having 6 to 10 carbon atoms which may have a substituent; OR⁰⁴ is a hydrogen atom, an alkyl group having 1 to 10 carbon atoms which may have a substituent, or an aryl group having 6 to 10 carbon atoms which may have a substituent; and OR⁰⁵ is a hydrogen atom, an alkyl group having 1 to 10 carbon atoms which may have a substituent, or an aryl group having 6 to 10 carbon atoms which may have a substituent.

In the general formula (1), a halogeno group denoted as E is, for example, a fluorine atom, a chlorine atom, a bromine atom, an iodine atom, or the like, preferably a fluorine atom, a chlorine atom, or bromine atom, and more preferably a chlorine atom. Examples of halogeno groups of R⁰¹, R⁰² and R⁰³ include those same as for E.

In the general formula (1), each of R⁰¹, R⁰² and R⁰³ is preferably a hydrogen atom, a chlorine atom, a cyano group, a carboxyl group, a methyl group, or a phenyl group, and more preferably a hydrogen atom, a chlorine atom, a cyano group, or a carboxyl group, and particularly preferably a hydrogen atom. R⁰¹, R⁰² and R⁰³ may be the same or different from one another, and a combination in which both R⁰² and R⁰³ are hydrogen atoms is particularly preferable.

Preferable examples of the comonomer include acrylonitrile, methacrylonitrile, acrylamide, methacrylamide, chloroacrylonitrile, vinyl chloride, vinyl bromide, 1,1-dichloroethylene, 2,3-dichloro-1-propene, acrylic acid, methacrylic acid, acrolein, methacrolein, vinylphosphonic acid, vinylsulfonic acid, itaconic acid, maleic acid, maleimide, maleic monoamide, monoethyl maleate, fumaric acid, fumaramide, monoethyl fumarate, fumaronitrile, N-(hydroxymethyl)acrylamide, N-(n-butoxymethyl)acrylamide, vinylalkoxysilanes such as vinyltrimethoxysilane, styrenesulfonic acid or a salt thereof, styrenesulfonic acid ester, and vinyl benzoate or a salt thereof.

One comonomer may be used, and plural comonomers may be used in combination.

The comonomer is preferably at least one crosslinkable comonomer selected from the group consisting of a comonomer in which E is a cyano group, a comonomer in which E is a formyl group, and a comonomer in which E is a carbamoyl group in the general formula (1). Use of such a crosslinkable comonomer allows effective generation of intermolecular and/or intramolecular crosslinking via a side chain of a polymer complex, and makes it possible to obtain a modified polymer complex particularly excellent in heat stability.

Preferable examples of a crosslinkable comonomer include acrylonitrile, methacrylonitrile, acrylamide, methacrylamide, chloroacrylonitrile, acrolein, methacrolein, maleimide, maleic monoamide, fumaraide, and fumaronitrile. Among these, acrylonitrile, methacrylonitrile, acrylamide, methacrylamide, and chloroacrylonitrile are more preferable. Use of such a crosslinkable comonomer allows effective generation of imine-type intermolecular and/or intramolecular crosslinking via a side chain of a polymer complex.

The comonomer is preferably at least one hydrophilic comonomer selected from the group consisting of acrylic acid, an acrylate, methacrylic acid, a methacrylate, vinylphosphonic acid, vinylsulfonic acid, vinylsulfonic acid ester, styrenesulfonic acid, styrenesulfonate, and sulfonic acid ester. As the vinylsulfonic acid ester or the vinylsulfonic acid ester, one easily giving a sulfonic acid site due to hydrolysis is preferable. Use of such a hydrophilic comonomer allows exhibition of good dispersibility in a polar solvent, in particular, a solvent containing water, and a catalyst activity becomes excellent when the comonomer is used as a redox catalyst in the solvent, thus being preferable.

More preferable examples of the hydrophilic comonomer include acrylic acid, vinylphosphonic acid, styrenesulfonic acid, lithium styrenesulfonate, sodium styrenesulfonate, potassium styrenesulfonate, rubidium styrenesulfonate, cesium styrenesulfonate, methyl styrenesulfonate, ethyl styrenesulfonate, propyl styrenesulfonate, and allyl styrenesulfonate. Among these, acrylic acid, styrenesulfonic acid, lithium styrenesulfonate, sodium styrenesulfonate, potassium styrenesulfonate, methyl styrenesulfonate, and ethyl styrenesulfonate are more preferable.

It is preferable to use a crosslinkable comonomer and a hydrophilic comonomer in combination as comonomers. A polymer complex and a modified polymer complex obtained by such a combination use have advantages derived from both of the crosslinkable comonomer and the hydrophilic comonomer and thus are excellent particularly in a catalyst ability to decompose hydrogen peroxide into water and oxygen.

When a crosslinkable comonomer and a hydrophilic comonomer are used in combination, a mixing ratio (crosslinkable monomer (weight)/hydrophilic monomer (weight)) is preferably within the range from 0.05 to 20, more preferably within the range from 0.1 to 10, and further more preferably within the range from 0.2 to 5. If the mixing ratio is within the above-described ranges, a polymer complex and a modified polymer complex having high heat stability and capable of being a redox catalyst excellent in a catalyst activity in the above-described polar solvent can be obtained.

### (Polymer Complex and Modified Polymer Complex)

Since a complex monomer has a polymerizable functional group having a polymerization ability, the complex monomer can be derived to a polymer complex by a polymerization reaction. The polymer complex may be formed by polymerizing the above-described complex monomer, or may be formed by copolymerizing the above-described complex and a comonomer such as a vinyl compound.

That is, when the complex monomer 1 is used, a complex polymer can be obtained by copolymerization of the complex monomer 1 and the comonomer. When the complex monomer 2 is used, a complex polymer can be obtained by mono-polymerization of the complex monomer 2, and a complex polymer can be also obtained by copolymerization of the complex monomer 2 and the comonomer described.

The above-described complex monomer and a comonomer are copolymerized to form a polymer complex and the polymer complex is subjected to intermolecular and/or intramolecular crosslinking via a reaction of its side chain (such a reaction may be called a “modification treatment”), and thus, a modified polymer complex can be obtained.

One embodiment of a structure of a modified polymer complex will be described using the case where a compound of the following formula (9) is used as a complex monomer, and the following formulas (1a) and (1b) are used as comonomers having a structure expressed by the general formula (1).

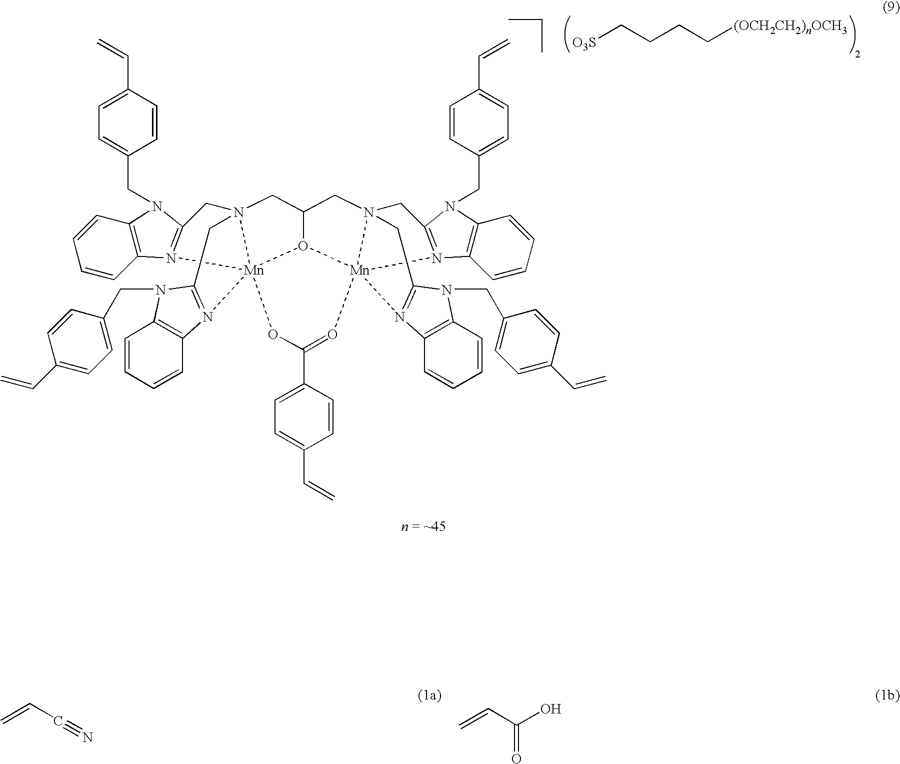

Since the polymer complex is a copolymer of a complex monomer of the formula (9) and comonomers of the following formulas (1a) and (1b), the chemical structure can be expressed by, for example, the following chemical formula (10). (Since the chemical formula (10) is a schematic view, a main chain and a part of side chain are omitted. In the chemical formula (10), a reaction product of a comonomer remaining with a polymer complex and a ligand is also shown.)

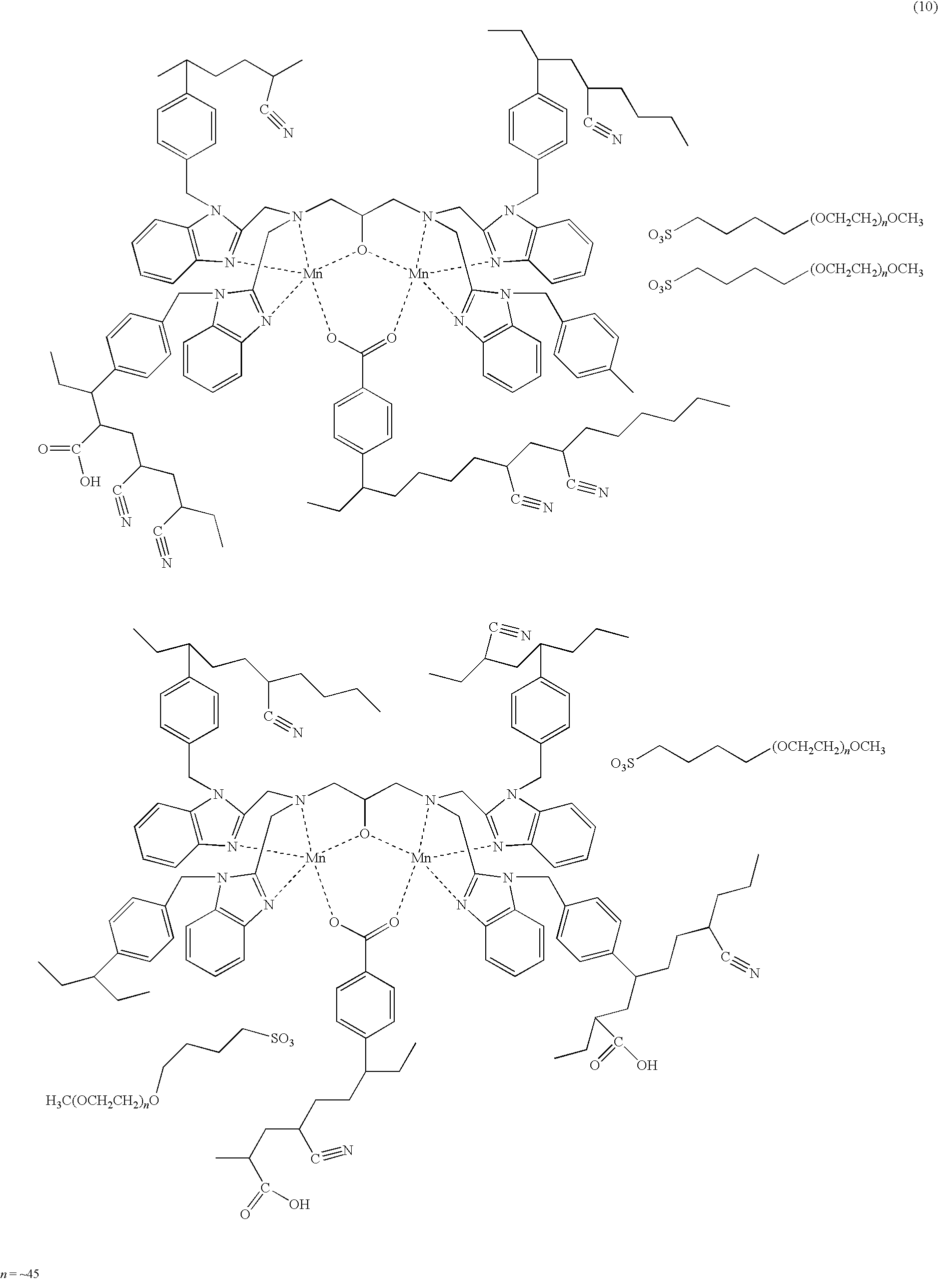

A polymer complex that can be expressed by the chemical formula (10) generates intermolecular crosslinking or intramolecular crosslinking via a reaction of its side chain by undergoing a modification treatment, for example, heating, and thus formed into a modified polymer complex. It is considered that any of flame retardant reactions listed in “Polymer Processing” (1993, Vol. 42, No. 12, p. 10, by Hideto Kakita, Koubunshikankoukai) occurs as a crosslinking reaction involving a cyano group among crosslinking reactions occurring in the modification treatment of the polymer complex, and any of flame retardant reactions listed in “Polymer Processing” (1993, Vol. 42, No. 12, p. 11-12, by Hideto Kakita, Koubunshikankoukai) occurs as a crosslinking reaction involving a cyano group and oxygen. A modified polymer complex obtained by a modification treatment of a polymer complex expressed by the chemical formula (10) has a chemical structure expressed by, for example, the following chemical formula (101). In the chemical formula (101), intramolecular crosslinking occurs in dashed circles denoted as L1, and intermolecular crosslinking occurs in dashed circles denoted as L2.

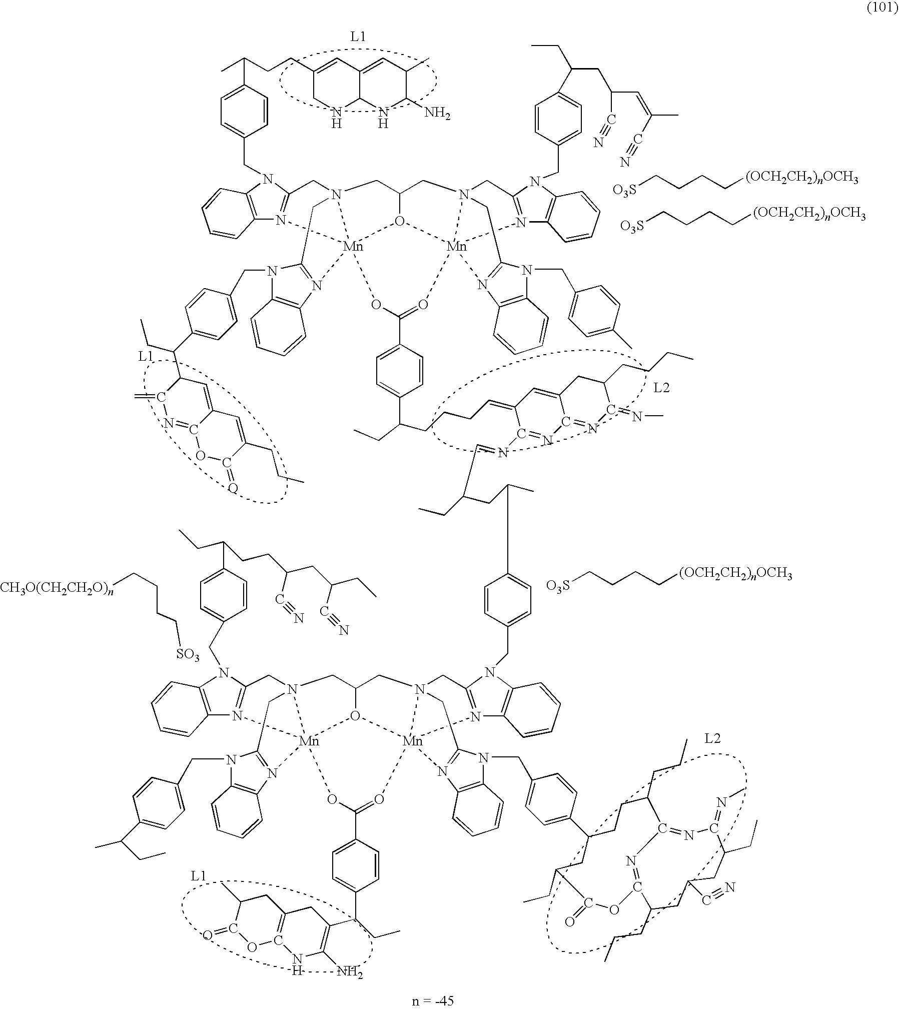

A polymerization reaction of a complex monomer or a copolymerization reaction of a complex monomer and a comonomer may be performed without solvent, or may be performed in the presence of a reaction solvent.

When the polymerization reaction or the copolymerization reaction is carried out using a reaction solvent, its reaction system may be a homogeneous system or a heterogeneous system. The polymerization reaction or the copolymerization reaction is operable in the presence of various solvents. Examples of the solvents include water, tetrahydrofuran, ether, 1,2-dimethoxyethane, acetonitrile, benzonitrile, acetone, methanol, ethanol, isopropanol, ethylene glycol, 2-methoxyethanol, 1-methyl-2-pyrrolidinone, dimethylformamide, dimethyl sulfoxide, acetic acid, hexane, pentane, benzene, toluene, xylene, dichloromethane, chloroform and carbon tetrachloride. These solvents may be used alone or in combination of two or more kinds.

The copolymerization is carried out by polymerizing at least one or more of the above-described complex monomers with at least one or more of comonomers. Thus, the copolymerization can be carried out by combining various polymerizable monomers in various monomer ratios.

As a polymerization initiation method of the polymerization or copolymerization, various techniques using heat, light, electrolysis, radiation, oxidation or the like can be used, and a radical generation catalyst, a radical initiator, etc. may be used. Among them, a polymerization initiation method using a radical initiator is preferable.

When polymerization or copolymerization using a radical initiator is performed, as the radical initiator, an organic peroxide such as benzoyl peroxide, an inorganic peroxide such as potassium persulfate, or an azo compound initiator such as 2,2′-azobis(2,4-dimethylvaleronitrile) can be used. The temperature of the polymerization or copolymerization is determined according to a radical generation temperature of the radical initiator used.

A form of the polymerization reaction of a complex monomer or a form of the copolymerization reaction of a complex monomer and a comonomer may be any of block polymerization, solution polymerization, suspension polymerization, emulsion polymerization, microemulsion polymerization, miniemulsion polymerization, precipitation polymerization, and dispersion polymerization. Preferable are suspension polymerization, emulsion polymerization, microemulsion polymerization, miniemulsion polymerization, precipitation polymerization, and dispersion polymerization, which can give a particulate polymer complex, and more preferable are emulsion polymerization, microemulsion polymerization, miniemulsion polymerization, and dispersion polymerization.

In a form of the polymerization reaction of a complex monomer or a form of the copolymerization reaction of a complex monomer and a comonomer, if necessity, additives may be used concomitantly, including water-soluble polymers such as polyvinyl alcohol, polyacrylic acid, polymethacrylic acid, gelatin, tragacanth, methyl cellulose, chitin, chitosan, and polymethacrylamide; polystyrene, polyacrylonitrile, polyaniline, polypyrrole, talc, bentonite, silica gel, diatom earth, clay, titanium oxide, BaSO₄, Al(OH)₃, CaSO₄, BaCO₃, MgCO₃, Ca(PO₄)₂, and CaCO₃; carbon additives such as fullerene, carbon black and active carbon; and nonionic surfactants, anionic surfactants, cationic surfactants and ampholytic surfactants, and these may be used alone or in combination of two or more kinds. As an additive, if necessary, a chain transfer agent can also be used concomitantly, including mercaptans such as t-dodecylmercaptan (TDM), n-dodecylmercaptan and n-octylmercaptan; α-methylstyrene dimer (αMSD) and terpinolenes (see JP-A No. 2005-54108).

The polymerization of a complex monomer or the copolymerization of a complex monomer and a comonomer is preferably performed in the presence of a carbon additive. The polymerization or copolymerization in the presence of a carbon additive enables to obtain a polymer complex and a modified polymer complex to which electrical conductivity derived from carbon and hydrophilicity and hydrophobicity derived from a functional group on a carbon surface are given, and use of a particulate carbon additive facilitates obtainment of a particulate polymer complex and a particulate modified polymer complex. Furthermore, when a carbon additive is used in inducing a polymer complex to a modified polymer complex, fusion between polymer complex particles can be suppressed, which makes obtainment of a particulate modified polymer complex comparatively easy.

In the present specification, a polymer complex obtained by polymerization or copolymerization in the presence of a carbon additive is also referred to as a polymer complex composite.

Examples of a carbon additive include carbon black, graphite, fullerene, and active carbon. Among these, carbon black is preferable.

Examples of carbon black include acetylene black, conductive furnace black (CF), super-conductive furnace black (SCF), extra-conductive furnace black (XCF), conductive channel black (CC), and furnace black or channel black which has been heat-treated at a high temperature of about 1500° C.

Specific examples of carbon black include Denka Acetylene Black (made by Denki Kagaku Kogyo K.K.), Shawnigan Acetylene Black (made by Shawnigan Chemical Co.), Continex CF (made by Continental Carbon Co.), Vulcan C (made by Cabot Corp.), Continex SCF (made by Continental Carbon Co.), Vulcan SC (made by Cabot Corp.), Asahi HS-500 (made by Asahi Carbon Co., Ltd.), Vulcan XC-7 (made by Cabot Corp.), Corax L (made by Degussa AG.), Ketjen Black EC and Ketjen Black EC-600JD (made by Ketjen Black International), a carbon nanopowder (made by Sigma-Aldrich Co.), nanom black ST (made by Frontier Carbon Corporation), and nanom mix ST (made by Frontier Carbon Corporation).

Preferable examples include Vulcan C (made by Cabot Corp.), Vulcan XC-7 (made by Cabot Corp.), Ketjen Black EC and Ketjen Black EC-600JD (made by Ketjen Black International), a carbon nanopowder (made by Sigma-Aldrich Co.), nanom black ST (made by Frontier Carbon Corporation), nanom mix ST (made by Frontier Carbon Corporation), and Aqua-black 001 (made by Tokai Carbon Co., Ltd.), and more preferable examples include Ketjen Black EC and Ketjen Black EC-600JD (made by Ketjen Black International).

When a polymer complex is synthesized by polymerizing the above-described complex monomer in the absence of carbon black, or when a polymer complex is synthesized by copolymerizing the complex monomer and a comonomer in the absence of carbon black, the obtained polymer complex preferably shows a peak in the wave number band from 2200 to 2300 cm⁻¹ in the IR spectrum, and preferably has a structure having a nitrile group corresponding to this peak. Specific examples of the structure having a nitrile group include a polyacrylonitrile structure, a polymethacrylonitrile structure, and a poly(chloroacrylonitrile) structure. When a polymer complex is synthesized by polymerizing the above-described complex monomer in the absence of carbon black or when a polymer complex is synthesized by copolymerizing the above-described complex monomer and a comonomer in the absence of carbon black, the obtained polymer complex preferably shows a peak in the wave number band from 3000 to 3550 cm⁻¹ in the IR spectrum, and preferably has a structure having a primary or secondary amide group corresponding to this peak. Specific examples of the structure having a primary or secondary amide group include a polyacrylamide structure, a polymethacrylonitrile structure, a poly(N-(hydroxymethyl)acrylamide) structure and a poly(N-(n-butoxymethyl)acrylamide) structure.

A polymer complex obtained by polymerizing a complex monomer (polynuclear complex) and a copolymer (polymer complex) obtained by copolymerizing a complex monomer and a comonomer (polymerizable monomer) preferably show molecular ionic peaks having m/Z of either 53 or 67 in a range from 400 to 500° C. as a thermogravimetric analysis condition in the thermogravimetric-mass spectrum measured under nitrogen gas flow. Here, m denotes the mass number of molecular ions, and Z denotes the charge number of molecular ions. It is considered that, in chains of such a polymer complex, a polyacrylonitrile-type structure or a polymethacrylonitrile-type structure is formed. Alternatively, it is considered that a polyacrylonitrile-type structure or a polymethacrylonitrile-type structure is formed in the modification treatment described later.

The polymer complex can be subjected to processing such as pulverization, if necessary. As the pulverization technique, pulverization by a mortar, an agate mortar, a ball mill, a jet mill, a fine mill, a disc mill, a hammer mill, and the like can be mentioned.

The obtained polymer complex is subjected to a modification treatment such as a heat treatment, a radiation irradiation treatment, an electromagnetic wave irradiation treatment or a discharge treatment, thereby leading the polymer complex to a modified polymer complex. A weight loss after the modification treatment is preferably 3% by weight or more and 50% by weight or less based on the weight before the modification treatment. Such a treatment can suppress decomposition of the complex structure and can lead to intermolecular and/or intramolecular crosslinking. The thus obtained modified polymer complex can be suitably used for a catalyst material having both a catalyst activity and good stability (heat resistance and acid resistance). Due to this modification treatment, a crosslinking reaction of a side chain derived from a complex comonomer and a comonomer remaining in the polymer complex occurs in a polymer chain (in the polymer complex), and it can be considered that this crosslinking reaction makes the complex structure rigid. It can be assumed that, due to this action, the modified polymer complex exhibits high stability when used as a catalyst. Mechanisms and actions of a crosslinking reaction occurring in the polymer complex are not limited thereto.

As the above-described modification treatment of a polymer complex, a heat treatment is preferable since it is simple and easy. The heat treatment can be performed under various conditions, and is preferably carried out under a temperature condition at which a crosslinking reaction occurs in a polymer chain (in the polymer complex) and decomposition of a complex structure occurs as less as possible. The temperature range of the heat treatment is preferably 150° C. or more and 1000° C. or less, more preferably 200° C. or more and 900° C. or less, furthermore preferably 250° C. or more and 600° C. or less, particularly preferably 300° C. or more and 500° C. or less, and more particularly preferably 300° C. or more and 400° C. or less.

Regarding a gas atmosphere in conducting the heat treatment, the treatment can be carried out under various gas atmospheres such as nitrogen, helium, argon, hydrogen, air, oxygen, carbon monoxide, water vapor and ammonia, and, nitrogen, helium and argon are preferable.

The modified polymer complex can be subjected to processing such as pulverization, if necessary. As the pulverization technique, pulverization by a mortar, an agate mortar, a ball mill, a jet mill, a fine mill, a disc mill, a hammer mill, and the like can be mentioned.

In addition, if a starting polymer complex is in a particulate form, a particulate modified polymer complex can be obtained without performing pulverization.

A polymer complex and a modified polymer complex are preferably in particulate forms; with regard to their particle diameters, an average particle diameter derived from a scanning electron micrograph is preferably 1 nm to 10 μm, more preferably 5 nm to 5 μm, further more preferably 10 nm to 1 μm, and particularly preferably 10 nm to 500 nm. When being in such a particulate form, a modified polymer complex is suitable for use as a redox catalyst. Such a particulate modified polymer complex has a large surface area and thus has a high reaction activity, and at the same time, introduction into a member becomes easy and the particulate modified polymer complex can be suitably used as a redox catalyst.

Conditions and a method of deriving an average particle diameter from a scanning electron micrograph will be shown below.

### [Conditions]

250 or more particles appear in a rectangular scanning electron micrograph, and the micrograph should be a scanning electron micrograph in which 15 or more particles can be confirmed in each divided image when the micrograph is evenly divided into 16 rectangular images.

### [Method]

Long diameters of three particles from each of the 16 divided images are measured, and an average of the long diameters of 48 particles measured is regarded as the average particle diameter. However, when at least one particle with a long diameter of more than 10 μm is present in the images, a long diameter of any one of the particles exceeding 10 μm in the images is regarded as an average particle diameter.

When a modified polymer complex is formed by performing a modification treatment on a polymer complex synthesized by copolymerizing a complex monomer and a comonomer in the absence of carbon black, the obtained modified polymer complex preferably shows peaks in the wave number bands from 1440 to 1390 cm⁻¹ and 1630 to 1590 cm⁻¹ in the IR spectrum in the infrared emission spectroscopy, and preferably has an imine crosslinking structure corresponding to this peak.

The polymer complex and the modified polymer complex preferably have at least one structure in which two transition metal atoms are coordinately bonded to the same coordinating atom is present. The modified polymer complex preferably has at least one structure in which a coordinating atom that is coordinately bonded to one transition metal atom and a coordinating atom that is coordinately bonded to another transition metal atom are bonded via 1 to 4 covalent bonds is present.

Due to such structures, a distance between two transition metal atoms becomes close, an interaction between the two transition metal atoms is easily exhibited, and thus, a catalyst activity of the modified polymer complex become higher.

Elemental compositions of the polymer complex and the modified polymer complex preferably contain 0.01 to 8% by weight of transition metals. Preferable examples of the transition metal are the same as described above. When the transition metal is in such a content, a catalyst activity is excellent when used as a redox catalyst, thus being preferable, and the complex is particularly excellent in an ability to decompose hydrogen peroxide into water and oxygen. When the content of the transition metal is large, the hydrogen peroxide decomposition catalyst ability is also improved, but polymerization reactivity in the above-described copolymerization reaction is lowered.

The content of the transition metal is more preferably 0.05 t 5% by weight, and further more preferably 0.1 to 4% by weight.

The content of the transition metal can be measured by an elemental analysis with ICP optical emission spectrometry.

The polymer complex and the modified polymer complex preferably have a gTOP defined by the Numerical Expression 1 below within the range from 1.8000 to 2.2400 in the solid electron spin resonance spectrum:

gTOP=hν/βH (Numerical Expression 1)

wherein h denotes a Planck constant, ν denotes a resonant frequency of a measured electromagnetic wave, β denotes a Bohr magneton, and H denotes a magnetic field intensity showing a maximum of an observed ESR signal, respectively.

The polymer complex and the modified polymer complex having such a gTOP have the above-described preferable metal central structure of the complex monomer containing a manganese atom.

The gTOP is more preferably within the range from 1.9000 to 2.2000, and further more preferably within the range from 1.9500 to 2.1000.

As described above, a complex monomer and a comonomer are copolymerized to form a polymer complex, and further, the polymer complex is derived to a modified polymer complex with a modification treatment. As a result, the modified polymer complex has both of heat resistance and acid resistant stability, and a unique catalyst activity of a polynuclear complex itself. When this modified polymer complex is used particularly as a hydrogen peroxide decomposition catalyst, not only it is possible to decompose hydrogen peroxide into water and hydrogen while suppressing the generation of free radicals, but also it is possible to have remarkably high heat stability as compared to conventional polynuclear complex catalysts, and thus the modified polymer complex can be preferably used as a redox catalyst.

The complex monomer, the polymer complex, and the modified polymer complex of the present embodiment and a redox catalyst using the same can be used for applications such as an antidegradant for polyelectrolyte type fuel cells and water electrolysis equipment, an antioxidant for medicines, agricultural chemicals and foods, etc.

Hereinafter, the present invention will be specifically described in reference to examples, but the present invention is not limited to the examples.

### Production Example 1

### Synthesis of Ligand

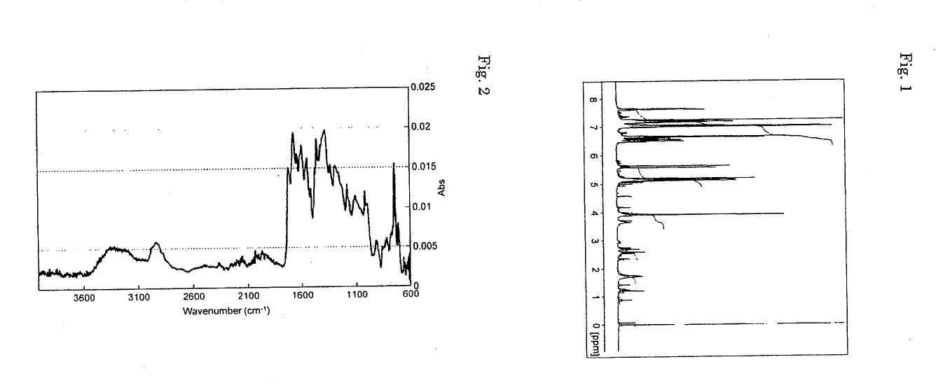

In accordance with the synthesis of an HL-Et ligand described in J. Am. Chem. Soc. 1984, 106, pp. 4765-4772, 2-hydroxy-1,3-diaminopropane tetraacetic acid was reacted with o-diaminobenzene, and then reacted with 4-chloromethylstyrene to obtain a bbpr-CH₂St ligand expressed by the following chemical formula (5) at a yield of 85%. This ligand was measured by ¹H-NMR (0.05% (v/v) TMS CDCl₃ solution) and as a result, introduction of a —CH₂St group was confirmed by a peak of 5 to 8 ppm. FIG. 1 shows a ¹H-NMR chart.

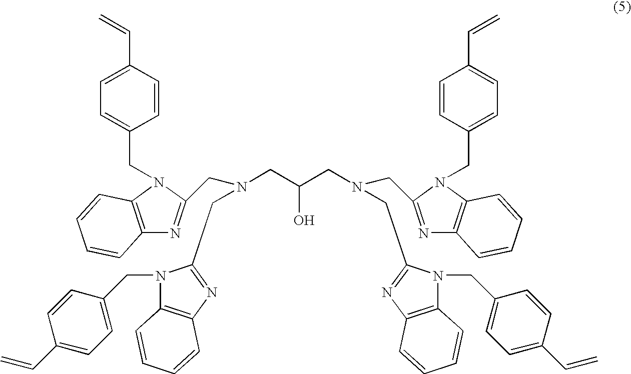

### Production Example 2

### Synthesis of Complex Monomer Precursor

Into a flask, p-vinylbenzoic acid (10.1 g, 67.5 mmol) and an aqueous NaOH solution (10.2 g, 64.1 mmol) were weighed, 140 ml of water was added thereto, the mixture was stirred to dissolve the solute, and the undissolved component was filtered off, thereby preparing an aqueous sodium p-vinylbenzoate solution. Separately, Mn(SO₄).5H₂O (7.74 g, 32.1 mmol) and 50 ml of water were weighed in a flask, and the mixture was stirred to dissolve the solute. The aqueous sodium p-vinylbenzoate solution was added thereto, and the mixture was stirred at room temperature for 2 hours. The precipitate produced was collected by filtration, washed with water, washed with ether, and then dried under reduced pressure to obtain white powder of manganese p-vinylbenzoate tetrahydrate (complex monomer precursor). The yield (amount) was 5.87 g (13.9 mmol), and the yield (rate) was 43%. Elemental analysis, Calculated for C₁₈H₂₂MnO₈: C, 51.32; H, 5.26. Found: C, 51.63; H, 5.16.

### Production Example 3

### Production of Complex Monomer

In a flask, bbpr-CH₂St (400 mg, 0.372 mmol) and NEt (i-Pr)₂ (43.2 mg, 0.335 mmol) were weighed, 54 ml of THF was added thereto, and the mixture was stirred to dissolve the solute. Manganese p-vinylbenzoate tetrahydrate (313 mg, 0.744 mmol) was added thereto, and the mixture was stirred at room temperature for 2 hours. This reaction mixture was concentrated under reduced pressure, and the precipitate produced by an addition of MeOH was collected by filtration, washed with water, washed with ether, and then dried under reduced pressure, thereby obtaining a beige powder of Mn-vb-(bbpr-CH₂St)-vb (complex monomer) expressed by the chemical formula (6) (same as the chemical formula (4a)). The yield was 122 mg. ESI MS, m/Z 1477.4 ([M-(p-vinylbenzoate anion)]⁺).

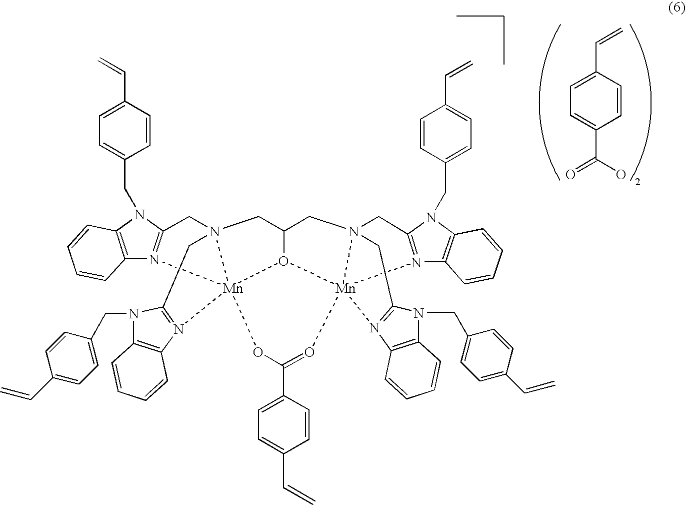

### Production Example 4

### Production of Polymer Complex

In a 10 ml sample tube made of glass, Mn-vb-(bbpr-CH₂St)-vb (200 mg), methacrylamide (60.0 mg), acrylic acid (49.3 mg), methacrolein (135 mg) and 2, 2′-azobis (2, 4-dimethylvaleronitrile) (18.4 mg) were mixed and dissolved. After an argon gas was flowed in this sample tube, the tube was sealed with a rubber septum, and the mixture was heated and polymerized on an oil bath of 50° C. for 11 hours. The polymer complex produced was taken out by crushing the sample tube. The polymer complex taken out was pulverized with a hammer and an agate mortar to obtain white powder (337 mg, the content of manganese is 0.731 μmol/mg provided that 100% of manganese at the start (at the time of polymerization initiation) is contained in the yield). An IR spectrum of the polymer complex obtained in Production Example 4 was measured. FIG. 2 shows the results. As shown in FIG. 2, the polymer complex obtained in Production Example 4 showed a peak in the wave number band from 3000 to 3550 cm⁻¹ in the IR spectrum, and thus, it was confirmed that the polymer complex had a polymethacrylamide structure.

### Example 1

### Production of Modified Polymer Complex

The polymer complex (107 mg) obtained in Production Example 4 was separated into two sample containers under the following conditions and heat-treated, and blackish brown powder of a modified polymer complex was obtained (59.7 mg, the content of manganese is 1.30 μmol/mg provided that 100% of manganese at the start is contained in the yield).
Apparatus: Rigaku TG8101D TAS200
Gas atmosphere: nitrogen, 200 ml/min.
Temperature condition: 40° C. to 350° C. (temperature rising speed: 10° C./min), then maintained at 350° C. for 15 min.
Sample container: open-type sample container made of aluminum (φ: 5.2, H: 5.0 mm, volume: 100 μl)
Amount of sample: 53±1 mg/the sample container

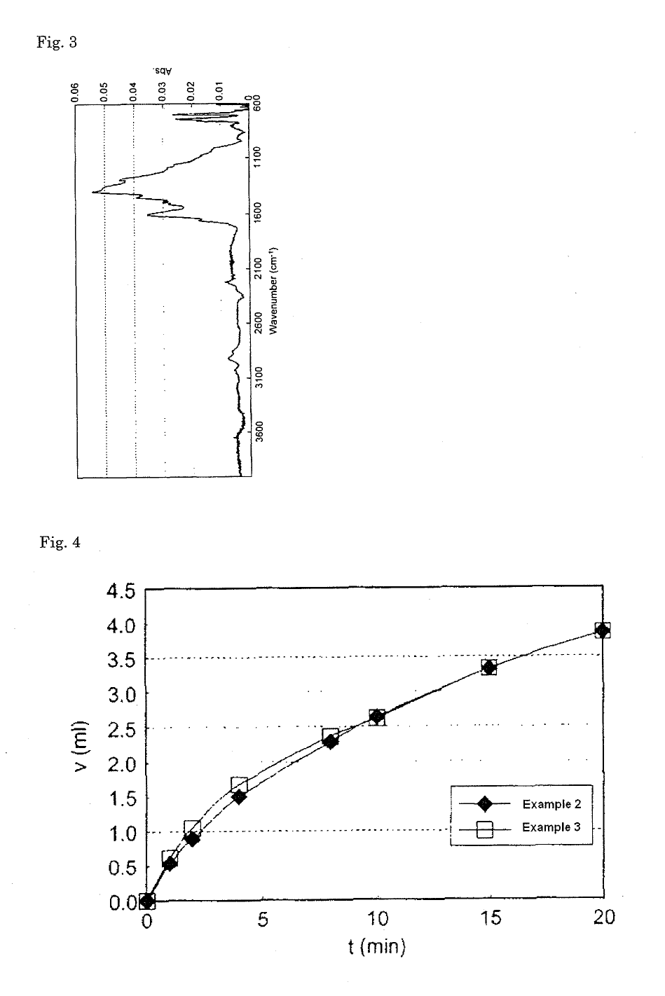

An IR spectrum of the modified polymer complex obtained in Example 1 was measured. FIG. 3 shows the results. As shown in FIG. 3, the modified polymer complex obtained in Example 1 showed a peak at 2220 cm⁻¹ in the IR spectrum, and thus, it was confirmed that an amide group in a polymethacrylamide chain contained in the polymer complex was changed to a nitrile group due to a modification treatment. The modified polymer complex obtained in Example 1 also showed peaks at 1602 cm⁻¹ and 1397 cm⁻¹, and thus, it can be considered that crosslinking involving the imine structure occurred in the modified polymer complex.

### [Thermogravimetric-Mass Spectrometric Analysis of Modified Polymer Complex]

When a thermogravimetric-mass spectrometric analysis was performed on the modified polymer complex obtained in Example 1 under the following conditions using the measurement apparatuses below, molecular ionic peaks having m/Z of 67, 77 and 78 were observed in a region from 400 to 500° C. in the thermogravimetric analysis.
Thermogravimetric analysis apparatus: TG-DTA6300 manufactured by SII NanoTechnology Inc.
Mass spectrometric analysis apparatus: QMS200 manufactured by PFEIFFER VACUUM, Inc.
Gas atmosphere: nitrogen (flow rate: 200 ml/min)
Temperature condition: 40° C. to 500° C. (temperature rising speed: 10° C./min)
Sample container: open-type sample container made of aluminum (φ: 5.2, H: 2.5 mm, volume: 50 μl)
Amount of sample: 2.1 mg/the sample container

### Example 2

### Hydrogen Peroxide Decomposition Test of Modified Polymer Complex

The modified polymer complex (6.35 mg, ca. 8.41 μmol (per one metal atom)) obtained in Example 1 was weighed into a 25 ml-two necked flask as a hydrogen peroxide decomposition catalyst. Thereto was added, as a solvent, a solution (1.00 ml) of poly(sodium 4-styrenesulfonate) (commercial product of Sigma-Aldrich Co., weight average molecular weight: about 70,000) dissolved in a tartaric acid/sodium tartarate buffer solution (prepared from a 0.20 mol/l aqueous tartaric acid solution and a 0.10 mol/l aqueous sodium tartarate solution, pH 4.0) to a polymer concentration of 21.1 mg/ml, and subsequently, ethylene glycol (1.00 ml) was added thereto and the mixture was stirred. The resultant solution was used as a catalyst mixed solution.

A septum was equipped to one inlet of the two necked flask containing this catalyst mixed solution, and the other inlet was connected to a gas bullet. After this flask was stirred at 80° C. for 5 minutes as a heat treatment before a reaction, an aqueous hydrogen peroxide solution (11.4 mol/l, 0.20 ml (2.28 mmol)) was added with a syringe, and a hydrogen peroxide decomposition reaction was conducted at 80° C. for 20 minutes. Oxygen being generated was measured with the gas burette, and the quantity of decomposed hydrogen peroxide was measured. In the determination of the quantity of hydrogen peroxide, the generated oxygen was measured with the gas burette, and the obtained measured volume (v) of oxygen was converted to obtain an amount of the generated gas (V) under the conditions taking the atmospheric pressure and a water vapor pressure into consideration (0° C., 101325 Pa (760 mmHg)) by the Numerical Expression 2. FIG. 4 shows a variation with time of the amount of generated oxygen (elapsed time is t).

V=[273 v (P−p)]/[760 (273+t)] (Numerical Expression 2)

(In the Numerical Expression 2, P: the atmospheric pressure (mmHg), p: vapor pressure of water (mmHg), t: temperature (° C.), v: measured volume (ml), V: volume (ml) at 0° C., 101325 Pa (760 mmHg).)

### Example 3

### Hydrogen Peroxide Decomposition Test of Modified Polymer Complex

A test of Example 3 was performed in the same manner as in Example 2, except for setting a temperature in the heat treatment before the reaction to 80° C. and stirring was performed for 24 hours. FIG. 4 also shows a variation with time of the converted amount of generated oxygen.

### Comparative Example 1

### Hydrogen Peroxide Decomposition Test of Mn-bbpr

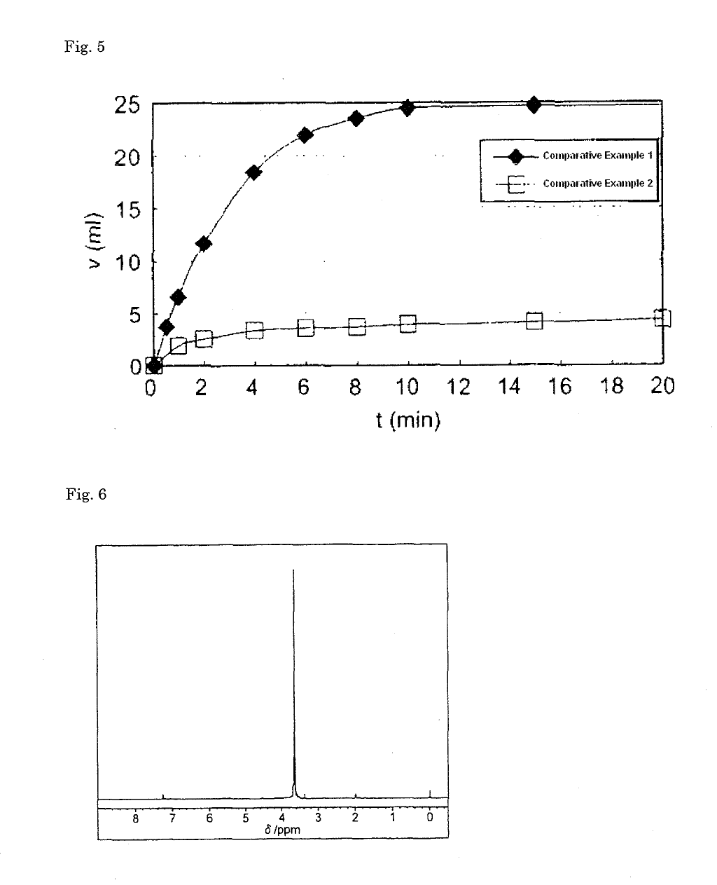

A test same as in Example 2 was performed using Mn-bbpr expressed by the following chemical formula (7) described in Patent Document 1 of the same metal molar quantity in place of the modified polymer complex obtained in Example 1 as a hydrogen peroxide decomposition catalyst. FIG. 5 shows a variation with time of the converted amount of generated oxygen.

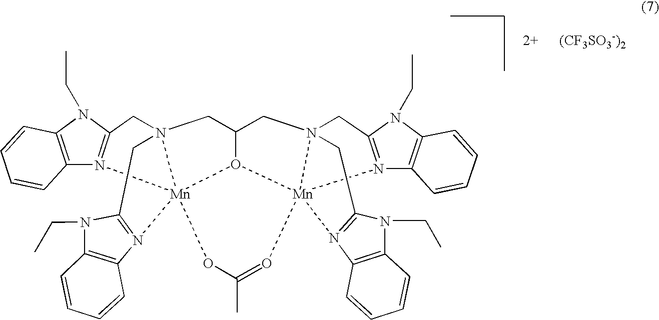

### Comparative Example 2

### Hydrogen Peroxide Decomposition Test of Mn-bbpr

A test of Comparative Example 2 was performed in the same manner as in Comparative Example 1 except for setting a temperature in the heat treatment before the reaction to 80° C. and stirring was performed for 24 hours. FIG. 5 also shows a variation with time of the converted amount of generated oxygen.

It was found that no decrease in a catalyst activity was shown in the modified polymer complex of the present invention shown as Examples 2 and 3 in FIG. 4 regardless of a time for a heat treatment before the reaction and the modified polymer complex had high heat stability. On the contrary, it was found that a catalyst activity largely decreased due to the heat treatment before the reaction for 24 hours in conventional Mn-bbpr catalysts shown as Comparative Examples 1 and 2 in FIG. 5, and that catalyst stability was low.

### Production Example 5

### Synthesis of Complex Monomer Precursor 3

Into a 500 ml three necked flask, polyethyleneglycolmonomethyl ester (73.4 g, Mn: 2000 or less), sodium hydroxide (36.7 mmol), and 1,4-butane sultone (36.7 mmol) were weighted, tetrahydrofuran (250 ml) was added thereto, and the mixture was stirred in an oil bath of 80° C. for 48 hours. Then, the solvent was distilled off under reduced pressure and dried in vacuum to obtain P₄₅C₄Na (complex monomer precursor) expressed by the following general formula (8) in a form of a brown solid. A yield of P₄₅C₄Na was 79.0 g. The P₄₅C₄Na was measured by ¹H-NMR (0.05% (v/v) TMS CDCL₃ solution). FIG. 6 shows the obtained ¹H-NMR chart. As shown in FIG. 6, introduction of a —CH₂CH₂CH₂SO₃Na group into P₄₅C₄Na (complex monomer precursor) was confirmed by a peak of 1.6 to 2.0 ppm and a peak of 2.8 to 2.9 ppm.

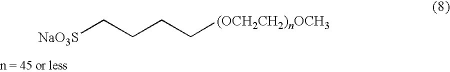

### Example 4

### Synthesis of Complex Monomer

Into a 200 ml flask, Mn-vb-(bbpr-CH₂St)-vb (1.00 g) and P₄₅C₄Na (2.67 g) were weighed, THF (60 ml) was added thereto and the mixture was stirred under reflux in an oil bath of 80° C. for 2 hours. Then, the solvent was distilled off from the reaction mixture under reduced pressure and washed with hexane to obtain Mn-vb-(bbpr-CH₂St)-P₄₅C₄ (complex monomer) expressed by the following chemical formula (9) in a form of ocher powder. The yield was 3.66 g.

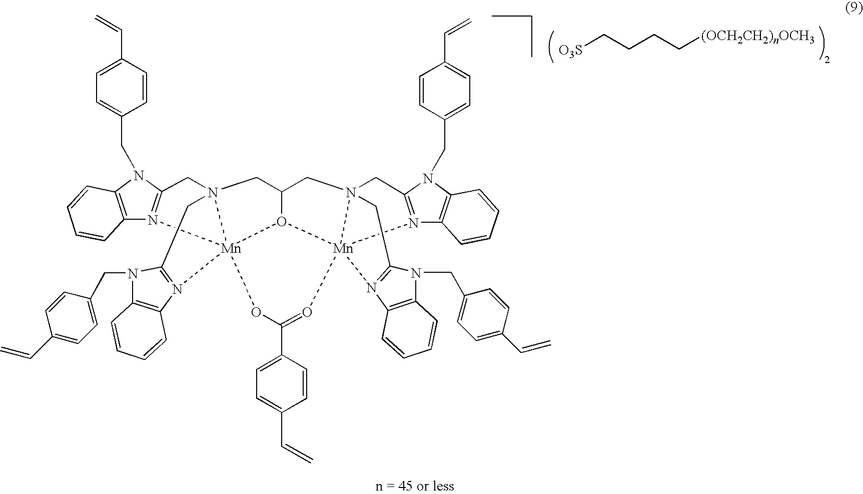

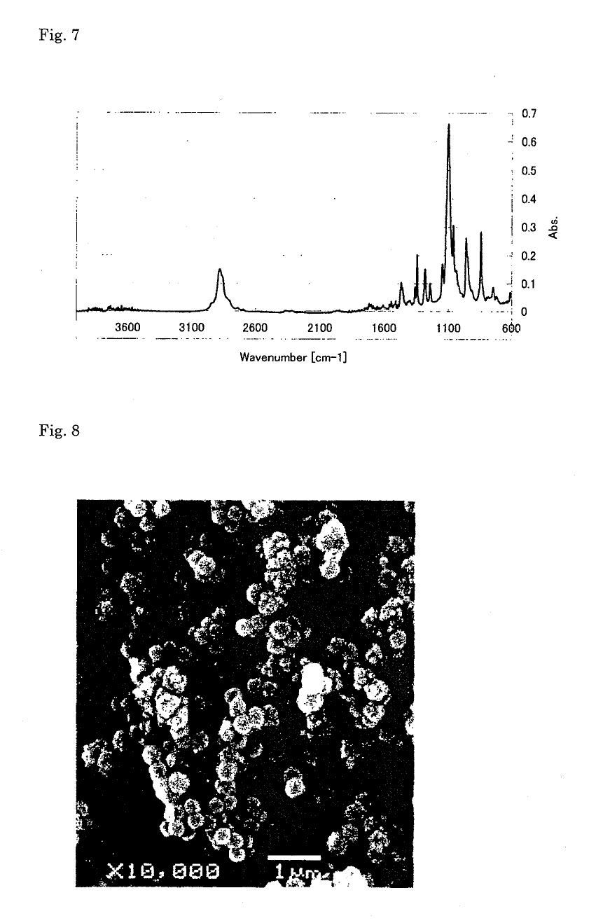

An infrared spectroscopic measurement of the complex monomer obtained in Example 4 was performed. FIG. 7 shows a spectrum chart.

A solid electron spin resonance spectrum of the complex monomer obtained in Example 4 was taken at −150° C. The gTOP was calculated from the above-described Numerical Expression 1 and found to be 2.0076.

### Example 5

### Production of Polymer Complex

To a 50 ml sample tube made of glass in which a glass-coated stirrer (φ 6 mm, L 25 mm), Mn-vb-(bbpr-CH₂St)-P₄₅C₄ (150 mg), ethanol (1.5 g), acrylonitrile (100 mg), acrylic acid (18.4 g), 2,2′-azobis(2,4-dimethylvaleronitrile) (10.0 mg), and water (1.5 g) were added and mixed with stirring. The atmosphere in this sample tube was replaced with a nitrogen gas, sealed with a rubber septum, and in such a state, the reaction mixture was heated and reacted at a rotation speed of 350 rpm for 1 hour using an oil bath of 60° C. and a magnetic stirrer. The generated polymer complex was filtered off from the reaction mixture, washed with methanol and then washed with ether, and dried in vacuum to obtain a polymer complex as white powder (73.7 mg). FIG. 8 shows a scanning electron micrograph of the obtained polymer complex. When an average particle diameter was derived from the scanning electron micrograph in FIG. 8 by the above-described method, the polymer complex was confirmed to be particles with an average particle diameter of 362 nm.

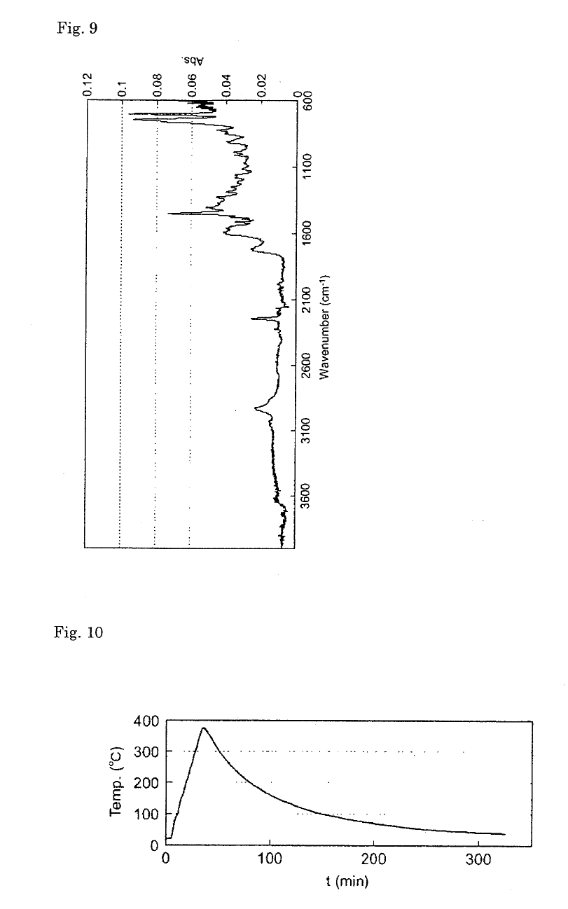

An IR spectrum of the polymer complex obtained in Example 5 was measured. FIG. 9 shows the results. The polymer complex obtained in Example 5 showed a peak in the wave number band of 2240 cm⁻¹ in the IR spectrum, and thus, it was confirmed that the polymer complex had a polyacrylonitrile structure having a cyano group.

### Example 6

### Production of Modified Polymer Complex

The polymer complex (50.0 mg) obtained in Example 5 was weighed in a 9 ml-sample tube made of glass, this sample tube was placed in the tubular furnace described below, and a nitrogen gas was flowed at a flow rate of 200 ml/min for 30 minutes. Then the polymer complex was heat-treated under the following temperature condition to obtain a modified polymer complex as a blackish brown powder (39.0 mg).
Apparatus: tubular furnace EPKRO-14K manufactured by Isuzu Seisakusho Co., Ltd.
Gas atmosphere: nitrogen, 200 ml/min.
Temperature condition: increasing from room temperature to 350° C. in 30 min., setting a device program in which the device power is off when reaching 350° C., thereafter naturally cooling to room temperature.

When an actual tubular furnace temperature was monitored, an excessive temperature increase of the apparatus was observed. FIG. 10 shows a variation with time of the tubular furnace temperature in the heat treatment.

### Example 7

### Production of Polymer Complex•Carbon Black Composite

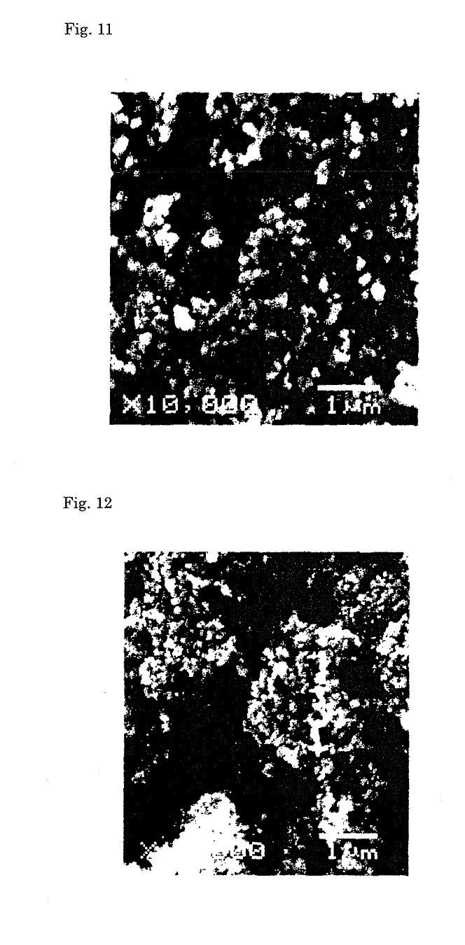

In a 50 ml sample tube made of glass, Mn-vb-(bbpr-CH₂St)-P₄₅C₄ (150 mg) was suspended in ethanol (1.5 g), 2,2′-azobis(2,4-dimethylvaleronitrile) (10 mg), acrylonitrile (113 mg), acrylic acid (24.3 mg), water (1.5 g) and Ketjen Black EC (50 mg) were sequentially added and mixed with stirring. After the atmosphere in this sample tube was replaced with nitrogen gas, the sample tube was sealed with a rubber septum and, in such a state, the reaction mixture was heated and reacted at a rotation speed of 350 rpm for 1 hour using an oil bath of 60° C. and a magnetic stirrer. After the reaction, an undissolved component in the sample tube was collected by filtration, washed with methanol, then washed with diethyl ether, and dried in vacuum to obtain a polymer complex•carbon black composite as black powder (87 mg). FIG. 11 shows a scanning electron micrograph of the obtained polymer complex•carbon black composite. When an average particle diameter was derived from the scanning electron micrograph in FIG. 11 by the above-described method, it was confirmed that the polymer complex-carbon black composite was particles with an average particle diameter of 179 nm.

### Example 8

### Production of Modified Polymer Complex

The polymer complex (72.1 mg) obtained in Example 7 was heat-treated by the same technique as in Example 6 to obtain a modified polymer complex as blackish brown powder (59.5 mg).

FIG. 12 shows a scanning electron micrograph of the obtained modified polymer complex. It was confirmed from the scanning electron micrograph in FIG. 12 that the modified polymer complex was particles with an average particle diameter of 119 nm.

### Example 9

### Production of Polymer Complex•Carbon Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), acrylonitrile (103 mg), acrylic acid (19 mg), dimethylformamide (1.2 g), water (1.8 g) and a carbon nanopowder (50 mg, made by Sigma-Aldrich Co.) as synthesis reagents to obtain a polymer complex•carbon composite (polymer complex composite) as black powder. The yield was 102 mg.

A solid electron spin resonance spectrum of the polymer complex composite obtained in Example 9 was measured at room temperature. The gTOP was calculated from the above-described (Numerical Expression 1) and found to be 1.9985.

### Example 10

### Production of Polymer Complex•Polyaniline•Carbon Black Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), acrylonitrile (101 mg), acrylic acid (18 mg), ethanol (1.5 g), water (1.5 g) and a polyaniline•carbon black composite (50 mg, 20% polyaniline by weight, made by Sigma-Aldrich Co.) as synthesis reagents to obtain a polymer complex•polyaniline•carbon black composite (polymer complex composite) as black powder. The yield was 80 mg.

### Example 11

### Production of Polymer Complex•Polypyrrole•Carbon Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), acrylonitrile (107 mg), acrylic acid (17 mg), ethanol (1.5 g), water (1.5 g) and a polypyrrole•carbon composite (50 mg, 20% polypyrrole by weight, made by Sigma-Aldrich Co.) as synthesis reagents to obtain a polymer complex•polypyrrole•carbon composite (polymer complex composite) as black powder. The yield was 93 mg.

### Example 12

### Production of Polymer Complex•Melamine Resin•Carbon Black Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), acrylonitrile (100 mg), acrylic acid (16 mg), ethanol (1.5 g), water (1.5 g), a methyl ethyl ketone solution of an acrylated (melamine•formaldehyde copolymer) (30 mg, 80% by weight, made by Sigma-Aldrich Co.) and Ketjen Black EC (50 mg) as synthesis reagents to obtain a polymer complex•melamine resin•carbon black composite (polymer complex composite) as black powder. The yield was 94 mg.

### Example 13

### Production of Polymer Complex•Carbon Black Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), methacrylamide (70 mg), acrylic acid (50 mg), ethanol (3.0 g), and Ketjen Black EC (50 mg) as synthesis reagents to obtain a polymer complex•carbon black composite (polymer complex composite) as black powder. The yield was 70 mg.

### Example 14

### Production of Polymer Complex•Carbon Black Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), acrylonitrile (70 mg), vinylphosphonic acid (50 mg), ethanol (3.0 g), and Ketjen Black EC (50 mg) as synthesis reagents to obtain a polymer complex•carbon black composite (polymer complex composite) as a black powder. The yield was 82 mg.

### Example 15

### Production of Polymer Complex•Carbon Black Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), acrylonitrile (70 mg), methacrolein (25 mg), acrylic acid (25 mg), ethanol (3.0 g), and Ketjen Black EC (50 mg) as synthesis reagents to obtain a polymer complex•carbon black composite (polymer complex composite) as black powder. The yield was 72 mg.

### Example 16

### Production of Polymer Complex•Carbon Black Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), acrylonitrile (30 mg), N-methylolacrylamide (30 mg), acrylic acid (50 mg), ethanol (3.0 g), and Ketjen Black EC (50 mg) as synthesis reagents to obtain a polymer complex•carbon black composite (polymer complex composite) as black powder. The yield was 88 mg.

### Example 17

### Production of Polymer Complex•Carbon Black Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), acrylonitrile (30 mg), N-(n-butoxymethyl)acrylamide (30 mg), acrylic acid (50 mg), ethanol (3.0 g), and Ketjen Black EC (50 mg) as synthesis reagents to obtain a polymer complex•carbon black composite (polymer complex composite) as black powder. The yield was 89 mg.

### Example 18

### Production of Polymer Complex•Carbon Black Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), acrylonitrile (100 mg), sodium styrenesulfonate (20 mg), acrylic acid (18 mg), ethanol (1.5 g), water (1.5 g) and Ketjen Black EC (50 mg) as synthesis reagents to obtain a polymer complex•carbon black composite (polymer complex composite) as black powder. The yield was 110 mg.

### Example 19

### Production of Polymer Complex•Carbon Black Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), methyl methacrylate (51 mg), 4-vinylpyridine (50 mg), acrylic acid (17 mg), ethanol (3.0 g), and Ketjen Black EC (50 mg) as synthesis reagents to obtain a polymer complex•carbon black composite (polymer complex composite) as black powder. The yield was 82 mg.

### Example 20

### Production of Polymer Complex•Carbon Black Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), N-vinylimidazole (55 mg), N-vinylpyrrolidone (53 mg), acrylic acid (19 mg), ethanol (3.0 g), and Ketjen Black EC (50 mg) as synthesis reagents to obtain a polymer complex•carbon black composite (polymer complex composite) as black powder. The yield was 82 mg.

### Example 21

### Production of Polymer Complex•Carbon Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), acrylonitrile (101 mg), acrylic acid (16 mg) methanol (3.0 g), and nanom mix ST (50 mg, made by Frontier Carbon Corporation) as synthesis reagents to obtain a polymer complex•carbon composite (polymer complex composite) as black powder. The yield was 66 mg.

### Example 22

### Production of Polymer Complex•Carbon Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), acrylonitrile (105 mg), acrylic acid (15 mg), methanol (3.0 g) and nanom black ST (50 mg, made by Frontier Carbon Corporation) as synthesis reagents to obtain a polymer complex•carbon composite (polymer complex composite) as black powder. The yield was 66 mg.

### (Example 23

### Production of Polymer Complex•Chitosan Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), acrylonitrile (102 mg), acrylic acid (17 mg), methanol (3.0 g), and a chitosan low molecular weight compound (50 mg, made by Sigma-Aldrich Co.) as synthesis reagents to obtain a polymer complex•chitosan composite (polymer complex composite) as milky white powder. The yield was 78 mg.

### Example 24

### Production of Polymer Complex•Carbon Black Composite

A reaction and a purification operation were carried out in the same manner as in Example 7 except for using Mn-vb-(bbpr-CH₂St)-P₄₅C₄ (150 mg) 2,2′-azobis(2,4-dimethylvaleronitrile) (10 mg), acrylonitrile (52 mg), vinyltrimethoxysilane (66 mg), anhydrous methanol (3.0 g), and Ketjen Black EC (50 mg) as synthesis reagents to obtain a polymer complex•carbon black composite (polymer complex composite) as black powder. The yield was 73 mg.

### Example 25

### Production of Polymer Complex•Carbon Black Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), acrylonitrile (21 mg), acrylic acid (20 mg), 2-propenyloxazoline (80 mg), methanol (3.0 g), and Ketjen Black EC (50 mg) as synthesis reagents to obtain a polymer complex•carbon black composite (polymer complex composite) as black powder. The yield was 71 mg.

### Example 26

### Production of Polymer Complex•Carbon Black Composite

A reaction and a purification operation were carried out in the same manner as in Example 7 except for using Mn-vb-(bbpr-CH₂St)-P₄₅C₄ (150 mg) 2,2′-azobis(2,4-dimethylvaleronitrile) (10 mg), 2,3-dichloro-1-propene (111 mg), acrylic acid (30 mg), ethanol (3.0 g), and Ketjen Black EC (50 mg) as synthesis reagents to obtain a polymer complex•carbon black composite (polymer complex composite) as black powder. The yield was 70 mg.

### Example 27

### Production of Polymer Complex•Carbon Black Composite

A reaction and a purification operation were carried out in the same manner as in Example 7 except for using Mn-vb-(bbpr-CH₂St)-P₄₅C₄ (150 mg), 2,2′-azobis(2,4-dimethylvaleronitrile) (10 mg), 2-chloroacrylonitrile (101 mg), acrylic acid (27 mg), ethanol (3.0 g), and Ketjen Black EC (50 mg) as synthesis reagents to obtain a polymer complex•carbon black composite (polymer complex composite) as black powder. The yield was 100 mg.

### Production Example 6

### Synthesis of Complex Precursor

Into a 500 ml flask, bbpr-CH₂St (2.00 g) and iron (III) chloride hexahydrate (1.01 g) were weighed, dimethyl sulfoxide (300 ml) was added thereto and the mixture was stirred on an oil bath of 80° C. for 24 hours. Then, the reaction mixture was gradually added to water contained in a 1 L-beaker and stirred, the obtained precipitate was filtered with a Kiriyama funnel, and the residue was washed with water and dried in vacuum, to obtain Fe—Cl-(bbpr-CH₂St)-Cl expressed by the following chemical formula (11) in a form of light green powder. The yield was 779 mg.

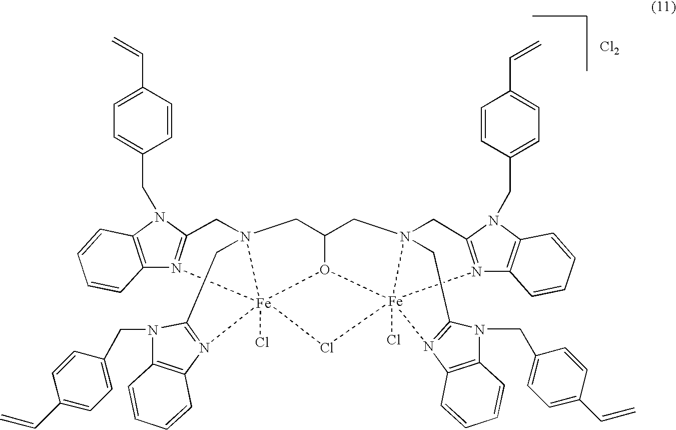

### Example 28

### Synthesis of Complex

Into a 100 ml flask, Fe—Cl-(bbpr-CH₂St)-Cl (300 mg) and P₄₅C₄Na (956 mg) were weighted, tetrahydrofuran (22 ml) was added thereto, and the mixture was heated under reflux on an oil bath of 80° C. for 27 hours. Then, the solvent was distilled off under reduced pressure to obtain Fe—Cl-(bbpr-CH₂St)-P₄₅C₄ expressed by the following chemical formula (12) in a form of yellowish brown powder. The yield was 1.18 g.

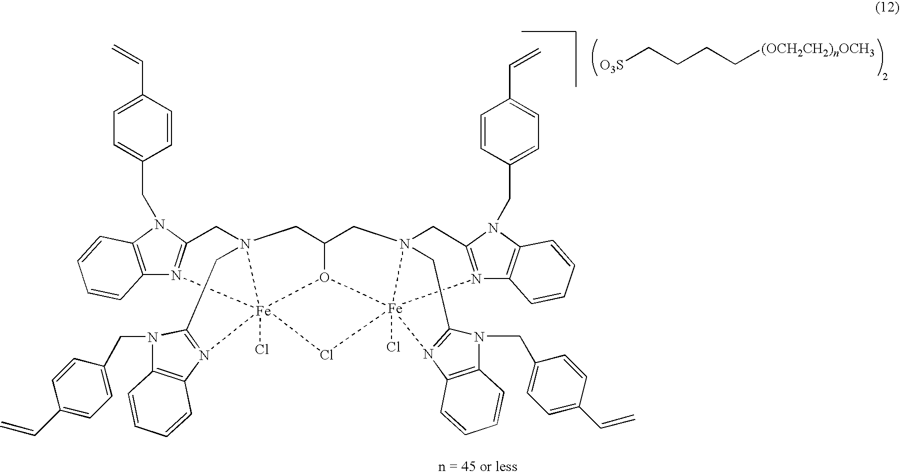

### Example 29

### Production of Polymer Complex•Carbon Black Composite

A reaction and a purification operation were carried out in the same manner as in Example 7 except for using Fe—Cl-(bbpr-CH₂St)-P₄₅C₄ (150 mg), 2,2′-azobis(2,4-dimethylvaleronitrile) (10 mg), acrylonitrile (106 mg), acrylic acid (21 mg), methanol (3.0 g), and Ketjen Black EC (50 mg) as synthesis reagents to obtain a polymer complex•carbon black composite (polymer complex composite) as black powder. The yield was 93 mg.

### Production Example 7

### Synthesis of Complex Precursor

Into a 500 ml flask, bbpr-CH₂St (2.00 g) and cobalt acetate tetrahydrate (927 mg) were weighed, dimethyl sulfoxide (300 ml) was added thereto and the mixture was stirred on an oil bath of 80° C. for 24 hours. Then, the reaction mixture was gradually added to water contained in a 1 L-beaker and stirred, the obtained precipitate was filtered off with a Kiriyama funnel, and the filtrate was washed with water and dried in vacuum, to obtain Co—OAc-(bbpr-CH₂St)-OAc expressed by the following chemical formula (13) in a form of orange powder. The yield was 688 mg. ESI MS, m/Z 625.2 ([M-2 (acetate anion)]²⁺).

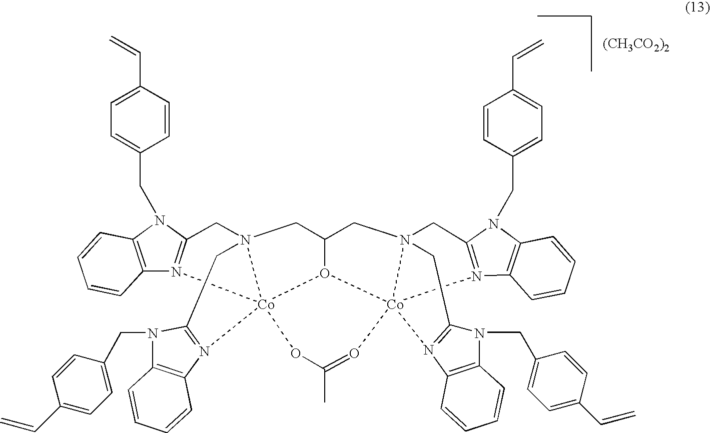

### Example 30

### Synthesis of Complex

In a 100 ml flask, Co—OAc-(bbpr-CH₂St)-OAc (300 mg) and P₄₅C₄Na (951 mg) were weighted, tetrahydrofuran (22 ml) was added thereto, and the mixture was heated under reflux on an oil bath of 80° C. for 27 hours. Then, the solvent was distilled off under reduced pressure to obtain Co—OAc-(bbpr-CH₂St)-P₄₅C₄ expressed by the following chemical formula (14) in a form of reddish brown powder. The yield was 1.19 g.

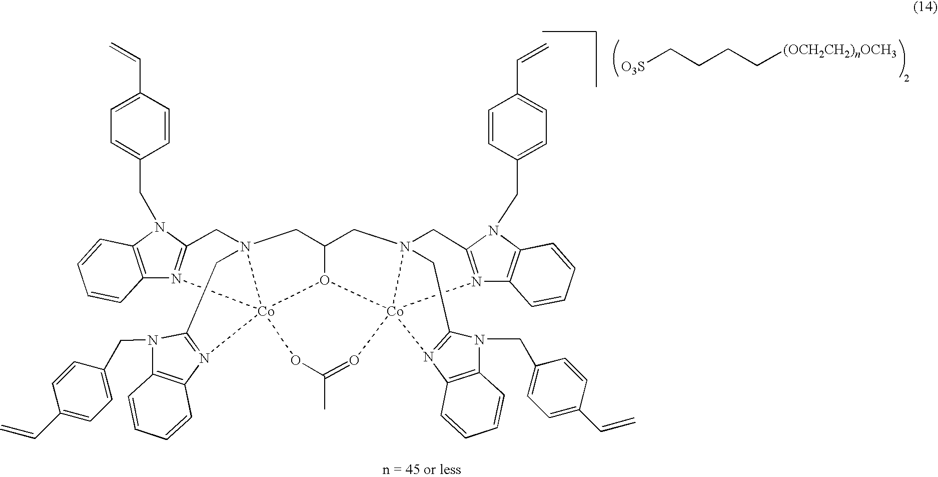

### Example 31

### Production of Polymer Complex•Carbon Black Composite

A reaction and a purification operation were carried out in the same manner as in Example 7 except for using Co—OAc-(bbpr-CH₂St)-P₄₅C₄ (150 mg), 2,2′-azobis(2,4-dimethylvaleronitrile) (10 mg), acrylonitrile (100 mg), acrylic acid (21 mg), methanol (3.0 g), and Ketjen Black EC (50 mg) as synthesis reagents to obtain a polymer complex•carbon black composite (polymer complex composite) as black powder. The yield was 94 mg.

### Production Example 8

### Synthesis of Complex Precursor

In a 500 ml flask, bbpr-CH₂St (2.00 g) and Ni—(OAc)₂.4H₂O (926 mg) were weighed, dimethyl sulfoxide (300 ml) was added thereto and the mixture was stirred on an oil bath of 80° C. for 24 hours. Then, the reaction mixture was gradually added to water contained in a 1 L-beaker and stirred, the obtained precipitate was filtered off with a Kiriyama funnel, and the filtrate was washed with water and dried in vacuum, to obtain Ni—OAc-(bbpr-CH₂St)-OAc expressed by the following chemical formula (15) in a form of yellow green powder. The yield was 937 mg. ESI MS, m/Z 624.2 ([M-2 (acetate anion)]²⁺).

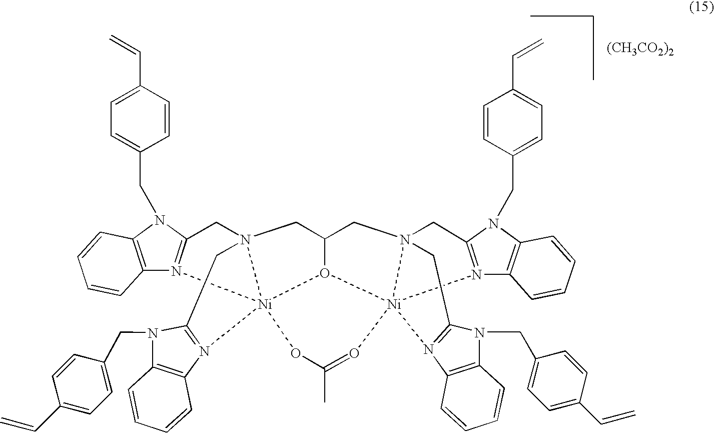

### Example 32

### Synthesis of Complex

In a 100 ml flask, Ni—OAc-(bbpr-CH₂St)-OAc (300 mg) and P₄₅C₄Na (951 mg) were weighted, tetrahydrofuran (22 ml) was added thereto and the mixture was heated under reflux in an oil bath of 80° C. for 27 hours. Then, the solvent was distilled off under reduced pressure to obtain Ni—OAc-(bbpr-CH₂St)-P₄₅C₄ expressed by the following chemical formula (16) in a form of greenish brown powder. The yield was 1.14 g.

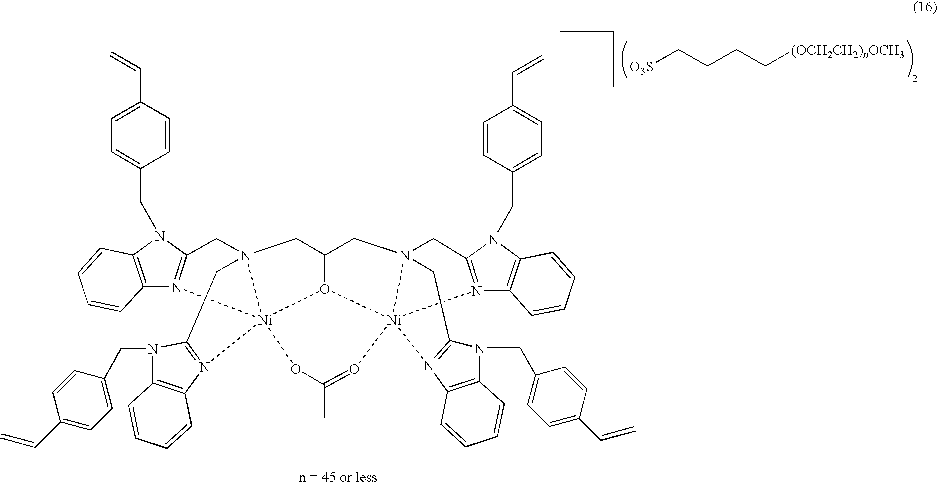

### Example 33

### Production of Polymer Complex•Carbon Black Composite

A reaction and a purification operation were carried out in the same manner as in Example 7 except for using Ni—OAc-(bbpr-CH₂St)-P₄₅C₄ (150 mg), 2,2′-azobis(2,4-dimethylvaleronitrile) (10 mg), acrylonitrile (103 mg), acrylic acid (23 mg), methanol (3.0 g), and Ketjen Black EC (50 mg) as synthesis reagents to obtain a polymer complex•carbon black composite (polymer complex composite) as black powder. The yield was 97 mg.

### Production Example 9

### Synthesis of Complex Precursor

Into a 500 ml flask, bbpr-CH₂St (2.00 g) and Cu(OAc)₂.H₂O (743 mg) were weighed, dimethyl sulfoxide (300 ml) was added thereto and the mixture was stirred on an oil bath of 80° C. for 24 hours. Then, the reaction mixture was gradually added to water contained in a 1 L-beaker and stirred, the obtained precipitate was filtered off with a Kiriyama funnel, and the filtrate was washed with water and hexane again in the same manner and dried in vacuum, to obtain Cu—OAc-(bbpr-CH₂St)-OAc expressed by the following chemical formula (17) in a form of brown powder. The yield was 1.14 g.

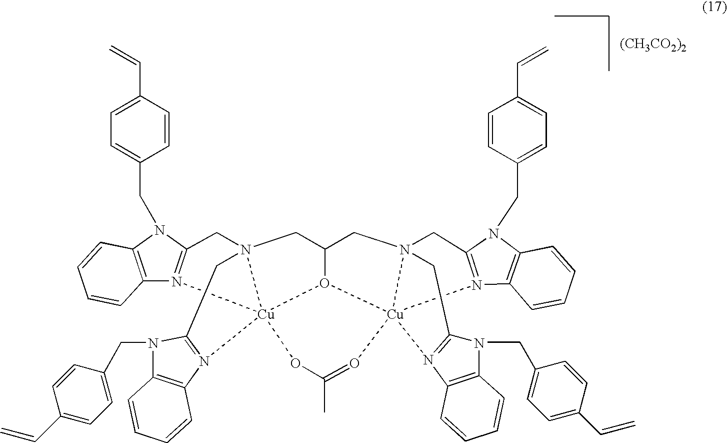

### Example 34

### Synthesis of Complex

Into a 100 ml flask, Cu—OAc-(bbpr-CH₂St)-OAc (300 mg) and P₄₅C₄Na (947 mg) were weighted, tetrahydrofuran (22 ml) was added thereto, and the mixture was heated under reflux on an oil bath of 80° C. for 27 hours. Then, the solvent was distilled off under reduced pressure to obtain Cu—OAc-(bbpr-CH₂St)-P₄₅C₄ expressed by the following chemical formula (18) in a form of brownish red powder. The yield was 1.22 g.

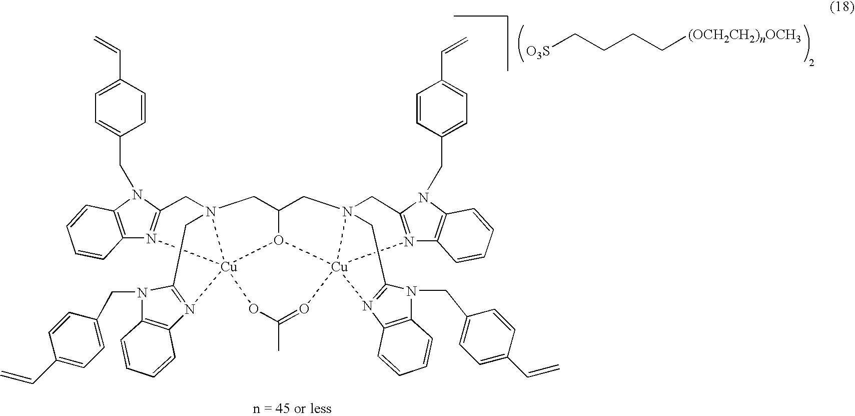

### Example 35

### Production of Polymer Complex•Carbon Black Composite

A reaction and a purification operation were carried out in the same manner as in Example 7 except for using Cu—OAc-(bbpr-CH₂St)-P₄₅C₄ (150 mg), 2,2′-azobis(2,4-dimethylvaleronitrile) (10 mg), acrylonitrile (103 mg), acrylic acid (18 mg), methanol (3.0 g), and Ketjen Black EC (50 mg) as synthesis reagents to obtain a polymer complex•carbon black composite (polymer complex composite) as black powder. The yield was 89 mg.

### Example 36

### Production of Polymer Complex•Carbon Black Composite

Into a 200 ml separable flask equipped with a mechanical stirrer, Mn-vb-(bbpr-CH₂St)-P₄₅C₄ (3.0 g), 2,2′-azobis(2,4-dimethylvaleronitrile) (200 mg), and distilled water (20 g) were added, thereto was then added a mixture obtained by previously ultrasonically stirring ethanol (30 ml) and Ketjen Black EC (1.0 g) in a 100 ml-vial, and the reaction mixture was stirred at 350 rpm at room temperature for 30 minutes. Thereto were added acrylonitrile (2.4 g), acrylic acid (400 mg), sodium p-styrenesulfonate (10 g), and distilled water (10 g), and the mixture was further stirred at 350 rpm at room temperature for 30 minutes. A nitrogen gas was bubbled in this flask for 30 minutes to bring the flask under a nitrogen gas flow, and then the mixture was heated and stirred at 350 rpm at 60° C. for 60 minutes to be reacted. After the reaction, the precipitate was collected by filtration, and washed with methanol. Then, the filtrated product was washed with a solution of methanol/water=9/1 (v/v), thereafter further washed with methanol, and the resultant was dried in vacuum to obtain a polymer complex•carbon black composite (polymer complex composite) as black powder. The yield was 4.69 g. The manganese content was measured with ICP optical emission spectrometry and found to be 0.38% by weight.

The solid electron spin resonance spectrum of the polymer complex obtained in Example 36 was measured at −150° C. When the gTOP was calculated from the Numerical Expression 1, it was found to be 1.9896, and it was confirmed that the polymer complex had a metal center of the starting manganese complex monomer.

### Examples 37 to 60

### Production of Modified Polymer Complex

Modified polymer complexes were obtained by respectively performing heat treatments by the same technique as in Example 6 to the polymer complexes obtained in Examples 9 to 27, 29, 31, 33, 35 and 36 (composites). Weights of the polymer complex composites used (amounts of starting polymer complex composites) and yields of the modified polymer complexes are collectively shown in Table 1.

TABLE 1

| | Starting polymer | Amounts of | | Yields of modified | |
|---|---|---|---|---|---|
| | complex composites | composites | | polymer complexes | |
| Example 37 | Example 9 | 84.6 | mg | 75.9 | mg |
| Example 38 | Example 10 | 72.0 | mg | 64.1 | mg |
| Example 39 | Example 11 | 84.1 | mg | 70.7 | mg |
| Example 40 | Example 12 | 87.0 | mg | 69.5 | mg |
| Example 41 | Example 13 | 67.9 | mg | 62.0 | mg |
| Example 42 | Example 14 | 76.0 | mg | 68.9 | mg |
| Example 43 | Example 15 | 68.1 | mg | 61.8 | mg |
| Example 44 | Example 16 | 82.0 | mg | 73.9 | mg |
| Example 45 | Example 17 | 97.5 | mg | 89.9 | mg |
| Example 46 | Example 18 | 78.8 | mg | 69.8 | mg |
| Example 47 | Example 19 | 73.6 | mg | 62.8 | mg |
| Example 48 | Example 20 | 73.9 | mg | 64.9 | mg |
| Example 49 | Example 21 | 58.3 | mg | 54.4 | mg |
| Example 50 | Example 22 | 72.4 | mg | 65.5 | mg |
| Example 51 | Example 23 | 74.8 | mg | 44.0 | mg |
| Example 52 | Example 24 | 65.4 | mg | 60.2 | mg |
| Example 53 | Example 25 | 62.2 | mg | 58.6 | mg |
| Example 54 | Example 26 | 68.5 | mg | 63.3 | mg |
| Example 55 | Example 27 | 92.5 | mg | 80.1 | mg |
| Example 56 | Example 29 | 91.2 | mg | 79.8 | mg |
| Example 57 | Example 31 | 90.3 | mg | 78.9 | mg |
| Example 58 | Example 33 | 88.6 | mg | 79.5 | mg |
| Example 59 | Example 35 | 80.0 | mg | 69.7 | mg |
| Example 60 | Example 36 | 3.67 | g | 3.28 | g |

The manganese content of the modified polymer complex obtained in Example 60 was measured with ICP optical emission spectrometry and found to be 0.42% by weight.

### Example 61

### Hydrogen Peroxide Decomposition Test of Modified Polymer Complex

The modified polymer complex (30.0 mg) obtained in Example 45 was weighed in a 25 ml-two necked flask as a hydrogen peroxide decomposition catalyst. Thereto was added, as a solvent, a tartaric acid/sodium tartarate buffer solution (prepared from a 0.20 mol/l aqueous tartaric acid solution and a 0.10 mol/l aqueous sodium tartarate solution, pH 4.0, 2.00 ml) and the mixture was stirred. The resultant solution was used as a catalyst mixed solution. The weight of the flask containing this catalyst mixed solution was measured before the reaction.

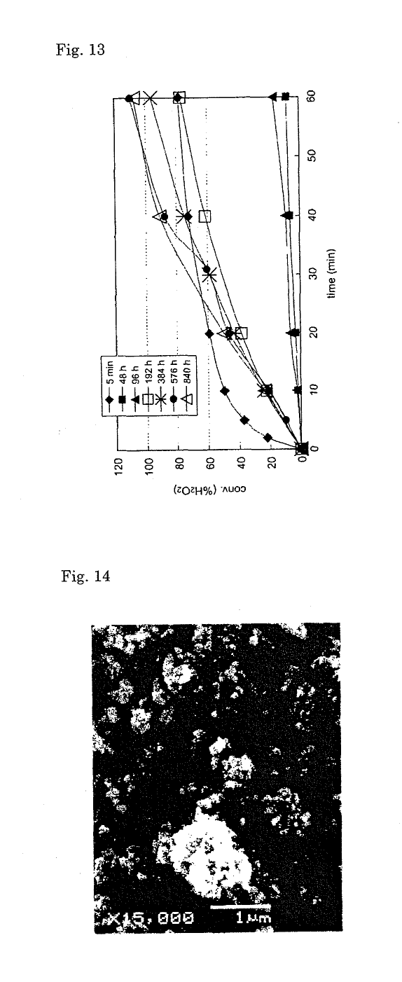

Next, a septum was equipped to one inlet of the two necked flask containing this catalyst mixed solution, and the other inlet was connected to a gas burette. After this flask was stirred at 80° C. for 5 minutes as a heat treatment before a reaction, an aqueous hydrogen peroxide solution (11.4 mol/l, 0.20 ml (2.28 mmol)) was added to the flask with a syringe, and a hydrogen peroxide decomposition reaction was conducted at 80° C. for 60 minutes. Oxygen being generated in this hydrogen peroxide decomposition reaction was measured with the gas burette, and the quantity of decomposed hydrogen peroxide was measured. In the same manner as in the above-described hydrogen peroxide decomposition test, the generated oxygen was measured with the gas burette, and the obtained measured volume (v) of oxygen was converted to obtain an amount of the generated gas (V) under the conditions taking the atmospheric pressure and a water vapor pressure into consideration (0° C., 101325 Pa (760 mmHg)). Then, a hydrogen peroxide decomposition rate and a variation with time of the hydrogen peroxide decomposition rate in process of the hydrogen peroxide decomposition reaction were found. The results are shown in FIG. 13. Note that the hydrogen peroxide decomposition rate was calculated assuming a hydrogen peroxide decomposition rate with V=25.5 ml to be 100%. In FIG. 13, the vertical axis shows a hydrogen peroxide decomposition rate (conv. (%) H₂O₂), the horizontal axis shows an elapsed time t (unit: minute), and the curve “5 min” shows a variation with time of hydrogen peroxide decomposition rates.

As described above, after the hydrogen peroxide decomposition reaction was performed at 80° C. for 60 minutes, the flask was continuously kept heated at 80° C. Then, at an each time point after a lapse of 48 h (hours), 96 h, 192 h, 384 h, 576 h, and 840 h from the time first starting the hydrogen peroxide decomposition reaction (hereinafter referred to as each elapsed time point), an aqueous hydrogen peroxide solution (11.4 mol/l, 0.20 ml (2.28 mmol)) was again added to the flask with a syringe. Then, each hydrogen peroxide decomposition reaction was made to proceed for further 60 minutes from the each elapsed time point, and a hydrogen peroxide decomposition rate and a variation with time of the hydrogen peroxide decomposition rates were found over further 60 minutes from the each elapsed time point in the same manner as in the above-described case. The results are shown in FIG. 13. Note that the curves “48 h,” “96 h,” “192 h,” “384 h,” “576 h” and “840 h” in FIG. 13 show variations with time of hydrogen peroxide decomposition rates over 60 minutes from each elapsed time point, respectively. In addition, when a series of the hydrogen peroxide decomposition reaction is proceeded, some water in the solvent was volatilized by keeping the flask heated at 80° C. for a long time, and thus, the weight of the flask containing the catalyst mixed solution was measured again 0.5 to 4 hours before the each elapsed time point from the start of the hydrogen peroxide decomposition reaction, and volatilized water was added to the flask, and the reaction was then performed.

It was confirmed from FIG. 13 that as compared to the case of the hydrogen peroxide decomposition reaction “5 min” initially performed, a catalyst activity of the modified polymer complex was once lowered in a hydrogen peroxide decomposition reaction starting from each elapsed time point of “48 h” and “96 h,” but improved in a hydrogen peroxide decomposition reaction starting from the elapsed time point of “192 h,” further improved in a hydrogen peroxide decomposition reaction carried out from the each elapsed time point of “384 h” and “576 h,” and was also high in a hydrogen peroxide decomposition reaction carried out from the elapsed time point of “840 h.” The results of this hydrogen peroxide decomposition reaction revealed that, in Example 61, the catalyst activity was improved due to an acid hot water treatment and a nonuniform catalyst (modified polymer complex) having both high heat resistance and high acid resistant stability was obtained.

### Example 62

### Hydrogen Peroxide Decomposition Test of Modified Polymer Complex

A catalyst (30 mg) prepared in the same manner as in Examples 7 and 8 was weighed in a 25 ml-two necked flask. Thereto was added, as a solvent, a solution (2.00 ml) of poly(sodium 4-styrenesulfonate) (commercial product of Sigma-Aldrich Co., weight average molecular weight: about 70,000) dissolved in a tartaric acid/sodium tartarate butter solution (prepared from a 0.20 mol/l aqueous tartaric acid solution and a 0.10 mol/l aqueous sodium tartarate solution, pH 4.0) to a polymer concentration of 10.5 mg/ml, and the mixture was stirred. The resultant solution was used as a catalyst mixed solution.

A septum was equipped to one inlet of the two necked flask containing this catalyst mixed solution, and the other inlet was connected to a gas burette. After this flask was stirred at 80° C. for 5 minutes as a heat treatment before a reaction, an aqueous hydrogen peroxide solution (11.4 mol/l, 0.20 ml (2.28 mmol)) was added with a syringe, and a hydrogen peroxide decomposition reaction was conducted at 80° C. for 20 minutes. Oxygen being generated was measured with the gas burette, and the quantity of decomposed hydrogen peroxide was measured. The measured volume (v) of the generated oxygen measured with the gas bullet was converted to obtain an amount of the generated gas (V) under the conditions taking the atmospheric pressure and a water vapor pressure into consideration (0° C., 101325 Pa (760 mmHg)) by the Numerical Expression 2. When a hydrogen peroxide decomposition rate with V=25.5 ml was assumed to be 100%, the hydrogen peroxide decomposition rate at this time was 39%.

Thereafter, the reaction solution was diluted with a water/acetonitrile mixed solution (water:acetonitrile=7:3, (v/v)) for the solution volume to be 10.0 ml, and this solution was filtered through a syringe filter. The filtrate was subjected to GPC measurement (GPC analysis conditions are as follows), and the weight average molecular weight of poly(sodium 4-styrenesulfonate) after the test was obtained. By comparing this weight average molecular weight after the test with the weight average molecular weight of poly(sodium 4-styrenesulfonate) before the test, the degree of reduction of a molecular weight of the polymer due to free radicals derived from hydrogen peroxide was examined, thereby estimating the amount of free radicals generated.

Table 2 shows the result of weight average molecular weights.

### GPC (Gel Permeation Chromatography) Analysis Conditions

Column: TSK gel a-M manufactured by Tosoh Corporation (13 μm, 7.8 mmφ×30 cm)

Column temperature: 40° C.

Mobile phase: 50 mmol/l aqueous ammonium acetate solution: CH₃CN=7:3 (v/v)

Flow rate: 0.6 ml/min.

Detector: RI

Injection amount: 50

Molecular weight calculation: a weight average molecular weight was obtained in terms of a polyethylene oxide conversion value.

[Measurement of Weight Average Molecular Weight of Poly(Sodium 4-styrenesulfonate) Before Test]

The weight average molecular weight of poly(sodium 4-styrenesulfonate) (commercial product of Sigma-Aldrich Co., weight average molecular weight: about 70,000) was measured in the same manner as the above-described GPC analysis conditions. Table 2 shows the results of the weight average molecular weights.

TABLE 2

| | Weight average |
|---|---|
| Samples | molecular weights |
| Example 62 | 1.0 × 10⁵ |
| Product before test | 1.0 × 10⁵ |

From Table 2, the weight average molecular weight of poly(sodium 4-styrenesulfonate) coexisted in Example 62 was almost the same as that in the product before the test. From this fact, it was made clear that the catalyst of Example 62 can suppress the generation of free radicals and decompose hydrogen peroxide.

### Example 63

### Production of Polymer Complex•Carbon Black Composite

To a 50 ml sample tube made of glass containing a glass-coated stirrer (φ 6 mm, L 25 mm), Mn-vb-(bbpr-CH₂St)-P₄₅C₄ (150 mg) and ethanol (1.5 g) were added and mixed, and 2,2′-azobis(2,4-dimethylvaleronitrile) (10 mg), acrylonitrile (100 mg), acrylic acid (one drop), water (1.5 g), and Ketjen Black EC (50 mg) were sequentially added and mixed with stirring. Three sample tubes containing such a reaction mixture were prepared, and each atmosphere of these sample tubes was replaced with a nitrogen gas, sealed with a rubber septum and, in such a state, each mixture was heated and reacted at a rotation speed of 350 rpm for 1 hour using an oil bath of 50° C. and a magnetic stirrer. After the reaction, undissolved components in the three sample tubes were unified together and collected by filtration, washed with methanol and then washed with diethyl ether, and dried in vacuum to obtain a polymer complex•carbon black composite (polymer complex composite) as a black powder. The yield was 293 mg. FIG. 14 shows a scanning electron micrograph of the obtained polymer complex•carbon black composite.

When an average particle diameter was derived with the above-described method, it was confirmed that the polymer complex•carbon black composite was particles with an average particle diameter of 179 nm.

### Example 64

### Hydrogen Peroxide Decomposition Test of Polymer Complex•Carbon Black Composite

The polymer complex•carbon black composite (10 mg) obtained in Example 63 was weighed into a 25 ml-two necked flask as a hydrogen peroxide decomposition catalyst (redox catalyst). Thereto was added, as a solvent, a tartaric acid/sodium tartarate butter solution (prepared from a 0.20 mol/l aqueous tartaric acid solution and a 0.10 mol/l aqueous sodium tartarate solution, pH 4.0, 2.00 ml) and the mixture was stirred. The resultant solution was used as a catalyst mixed solution (catalyst was insoluble).

A septum was equipped to one inlet of the two necked flask containing this catalyst mixed solution, and the other inlet was connected to a gas bullet. After this flask was stirred at 80° C. for 5 minutes as a heat treatment before the reaction, an aqueous hydrogen peroxide solution (11.4 mol/l, 0.20 ml (2.28 mmol)) was added with a syringe, and a hydrogen peroxide decomposition reaction was conducted at 80° C. for 60 minutes. Oxygen being generated was measured with the gas burette, and the quantity of decomposed hydrogen peroxide was measured. That is, in the determination of the quantity of hydrogen peroxide, the generated oxygen was measured with the gas burette and the obtained measured volume (v) of oxygen was converted to obtain an amount of the generated gas (V) under the conditions taking the atmospheric pressure and a water vapor pressure into consideration (0° C., 101325 Pa (760 mmHg)) by the following Numerical Expression 2.

V=[273 v (P−p)]/[760 (273+t)] (Numerical Expression 2)

(In the Numerical Expression 2, P: the atmospheric pressure (mmHg), p: vapor pressure of water (mmHg), t: temperature (° C.), v: measured volume (ml), V: volume (ml) at 0° C., 101325*Pa (760 mmHg).)

The results of the hydrogen peroxide decomposition test revealed that oxygen involving decomposition of hydrogen peroxide was generated over time and the oxygen generation of V=2.37 ml in 1 hour was observed. The fact made clear that the redox catalyst (nonuniform catalyst) of the present invention has a catalyst activity to decompose hydrogen peroxide.

### INDUSTRIAL APPLICABILITY

The modified polymer complex of the present invention can decompose hydrogen peroxide into water and oxygen with suppressing generation of free radicals particularly when used as a hydrogen peroxide decomposition catalyst, and also has remarkably high heat stability as compared to conventional polynuclear complex catalysts and is thus useful as a redox catalyst.

1. A modified polymer complex, which is obtained by intermolecular and/or intramolecular crosslinking of a polymer complex via side chains thereof, wherein the polymer complex is a copolymer of a complex monomer meeting the following conditions (i) to (iii) and a comonomer expressed by the following general formula (1):
(i) the complex monomer has two or more transition metal atoms;
(ii) the complex monomer has a polydentate ligand containing three or more coordinating atoms that are coordinately bonded to the transition metal atoms; and
(iii) the polydentate ligand has one or more polymerizable functional groups; 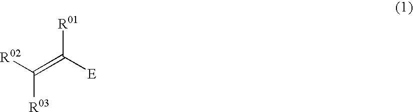
wherein E denotes a cyano group, a carboxyl group, a formyl group, a carbamoyl group, a phosphonic acid group, a sulfonic acid group, a halogeno group, a —CONHCH₂OR⁰⁴ group or a —Si(OR⁰⁵)₃ group, each of R⁰¹, R⁰² and R⁰³ independently denotes a hydrogen atom, a halogeno group, a cyano group, a —COOR⁰⁴ group, an alkyl group having 1 to 10 carbon atoms which may have a substituent, or an aryl group having 6 to 10 carbon atoms which may have a substituent; R⁰⁴ is a hydrogen atom, an alkyl group having 1 to 10 carbon atoms which may have a substituent, or an aryl group having 6 to 10 carbon atoms which may have a substituent; and R⁰⁵ is a hydrogen atom, an alkyl group having 1 to 10 carbon atoms which may have a substituent, or an aryl group having 6 to 10 carbon atoms which may have a substituent.

2. The modified polymer complex according to claim 1, wherein the transition metal atoms are transition metal atoms in the first transition element series.

3. The modified polymer complex according to claim 1, wherein at least one structure in which two transition metal atoms are coordinately bonded to the same coordinating atom is present.

4. The modified polymer complex according to claim 1, wherein at least one structure in which a coordinating atom that is coordinately bonded to one transition metal atom and a coordinating atom that is coordinately bonded to another transition metal atom are bonded via 1 to 4 covalent bonds is present.

5. The modified polymer complex according to claim 1, wherein the polydentate ligand has a structure expressed by the following general formula (2): 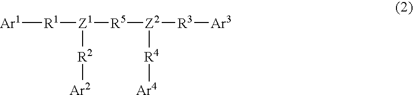
wherein each of Ar¹, Ar², Ar³ and Ar⁴ independently denotes an aromatic nitrogen-containing heterocyclic group, each of groups R¹, R², R³, R⁴ and R⁵ independently denotes a divalent group, and each of Z¹ and Z² independently denotes a nitrogen atom or a trivalent group; and at least one of Ar¹, Ar², Ar³, Ar⁴, R¹, R², R³, R⁴ and R⁵ has a polymerizable functional group.

6. The modified polymer complex according to claim 1, wherein the polydentate ligand has a structure expressed by the following general formula (3a) or (3b): 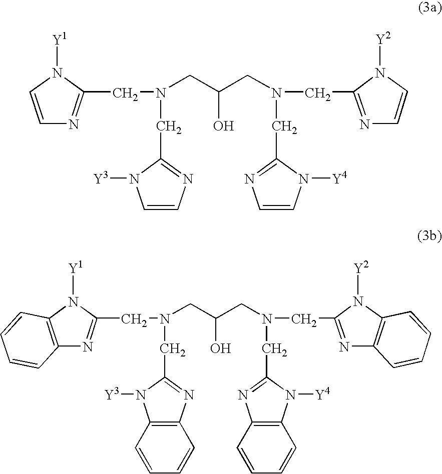
wherein each of Y¹, Y², Y³ and Y⁴ independently denotes a hydrogen atom, an alkyl group having 1 to 50 carbon atoms, or an aromatic group having 2 to 60 carbon atoms, and at least one of Y¹, Y², Y³ and Y⁴ is an alkyl group having 1 to 50 carbon atoms which has a polymerizable functional group, or an aromatic group having 2 to 60 carbon atoms which has a polymerizable functional group.

7. The modified polymer complex according to claim 1, wherein the comonomer contains at least one crosslinkable comonomer selected from a comonomer in which E is a cyano group, a comonomer in which E is a formyl group, and a comonomer in which E is a carbamoyl group in the general formula (1).

8. The modified polymer complex according to claim 1, wherein the comonomer contains at least one crosslinkable comonomer selected from the group consisting of acrylonitrile, methacrylonitrile, acrylamide, methacrylamide, and chloroacrylonitrile.

9. The modified polymer complex according to claim 1, wherein the comonomer contains at least one hydrophilic comonomer selected from the group consisting of acrylic acid, methacrylic acid, vinylphosphonic acid, vinylsulfonic acid, styrenesulfonic acid, a styrenesulfonate salt, and a styrenesulfonic acid ester.

10. The modified polymer complex according to claim 1, wherein the comonomer contains at least one of the crosslinkable comonomers and at least one of the hydrophilic comonomers.

11. The modified polymer complex according to claim 1, wherein the modified polymer complex is obtained by copolymerizing the complex monomer and the comonomer in the presence of a carbon additive.

12. The modified polymer complex according to claim 1, wherein the polymer complex shows a molecular ionic peak having m/Z of 53 or 67 when a mass number of a molecular ion is assumed to be m and a charge number of the molecular ion is assumed to be Z in a thermogravimetric-mass spectrum.

13. The modified polymer complex according to claim 1, which is obtained by intermolecular and/or intramolecular crosslinking of the polymer complex by a heat treatment, a radiation irradiation treatment, an electromagnetic wave irradiation treatment or a discharge treatment, wherein a weight loss after the treatment is 3% by weight or more and 50% by weight or less based on the weight before the treatment.

14. The modified polymer complex according to claim 1, which is obtained by intermolecular and/or intramolecular crosslinking of the polymer complex by a heat treatment at a temperature within the range from 200 to 900° C.

15. The modified polymer complex according to claim 1, which is in a particulate form having an average particle diameter derived from a scanning electron micrograph within the range from 10 nm to 10 μm.

16. The modified polymer complex according to claim 1, wherein a content of the transition metals is 8 to 0.01% by weight in an elemental analysis with an ICP optical emission spectrometry.

17. The modified polymer complex according to claim 1, wherein a peak maximum is shown within the ranges from 1390 to 1440 cm⁻¹ and 1590 to 1630 cm⁻¹ in an infrared spectroscopy.

18. The modified polymer complex according to claim 1, wherein a gTOP defined by the following (Formula 1) is within the range from 1.8000 to 2.2400 in a solid electron spin resonance spectrum:
gTOP=hν/βH (Formula 1)
wherein h denotes a Planck constant, ν denotes a resonant frequency of a measured electromagnetic wave, β denotes a Bohr magneton, and H denotes a magnetic field intensity showing a maximum of an observed ESR signal, respectively.

19. A complex monomer meeting the following conditions (i′) to (iv′):
(i′) the complex monomer has one or more transition metal atoms;
(ii′) the complex monomer has a polydentate ligand containing three or more coordinating atoms that are coordinately bonded to the transition metal atoms;
(iii′) the polydentate ligand has one or more polymerizable functional groups; and
(iv′) the complex monomer has any structure of an organic acid salt structure, an amine salt structure, an ammonium salt structure, a pyridinium salt structure, an imidazolium salt structure, a hydroxyl group structure, an ether structure, and an acid amide structure.

20. The complex monomer according to claim 19, comprising at least one of the functional groups expressed by the following general formulas (1-1), (1-2), (1-3), (1-4), (1-5), (1-6), (1-7), (1-8) and (1-9) in the structure of (iv′): 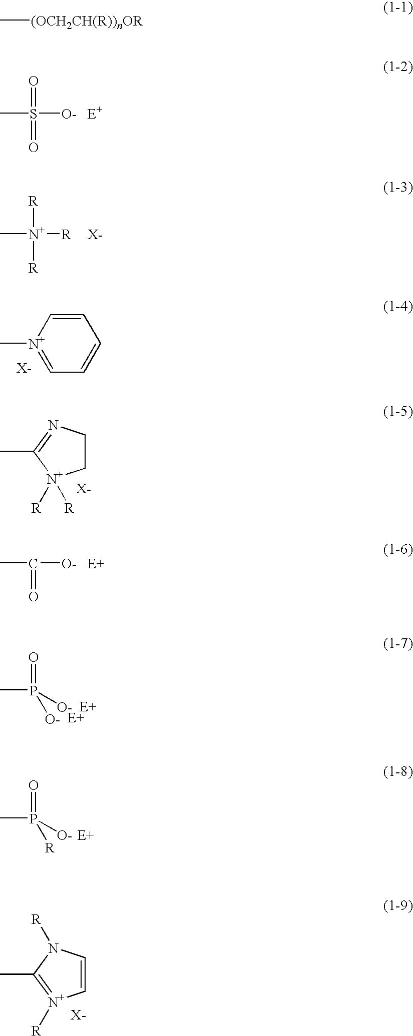
wherein n denotes an integer of 1 to 500, E⁺ denotes a proton, a lithium ion, a sodium ion, a potassium ion, a rubidium ion, a cesium ion or an ammonium ion, R denotes a hydrogen atom, an alkyl group having 1 to 50 carbon atoms which may have a substituent, or an aryl group having 6 to 50 carbon atoms which may have a substituent, and X″ denotes a fluoride ion, a chloride ion, a bromide ion, an iodide ion, a methanesulfonate ion, or a trifluoromethanesulfonate ion, respectively.

21. The complex monomer according to claim 19, wherein the transition metal atom is a transition metal atom in the first transition element series.

22. The complex monomer according to claim 19, having a structure expressed by the following general formula (2-1):
(L⁰¹)ₚ(M)ₘ(L⁰²)_{q} (2-1)
wherein M denotes a transition metal atom, m denotes an integer of 1 to 20, p denotes an integer of 1 to 5, and q denotes an integer of 1 to 20, respectively; L⁰¹ is a polydentate ligand having 3 or more atoms including a nitrogen coordinating atom, which has a substituent containing a polymerizable functional group or a functional group expressed by the general formula (1-1); and L⁰² is a ligand or a counter ion, which has a substituent containing a polymerizable functional group or a functional group expressed by the general formula (1-1), provided that a combination of the substituents in L⁰¹ and L⁰² is a combination of a polymerizable functional group and a functional group expressed by the general formula (1-1).

23. The complex monomer according to claim 19, comprising two or more transition metal atoms, wherein at least one structure in which two transition metal atoms among the two or more transition metal atoms are coordinately bonded to the same coordinating atom is present.

24. The complex monomer according to claim 19, comprising two or more transition metal atoms, wherein at least one structure in which a coordinating atom that is coordinately bonded to one transition metal atom among the two or more transition metal atoms and a coordinating atom that is coordinately bonded to a transition metal atom other than the one transition metal atom among the two or more transition metal atoms are bonded via 1 to 4 covalent bonds is present.

25. The complex monomer according to claim 22, wherein L⁰¹ in the general formula (2-1) has a structure expressed by the following general formula (2): 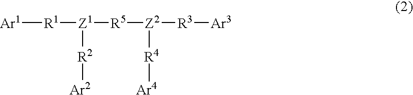
wherein each of Ar¹, Ar², Ar³ and Ar⁴ independently denotes a nitrogen-containing aromatic heterocyclic group, each of R¹, R², R³, R⁴ and R⁵ independently denotes a divalent group, and each of Z¹ and Z² independently denotes a nitrogen atom or a trivalent group, respectively; and at least one of Ar¹, Ar², Ar³, Ar⁴, R¹, R², R³, R⁴ and R⁵ has one polymerizable functional group.

26. The complex monomer according to claim 22, having a structure in which L⁰¹ in the general formula (2-1) is expressed by the following general formula (3a) or (3b): 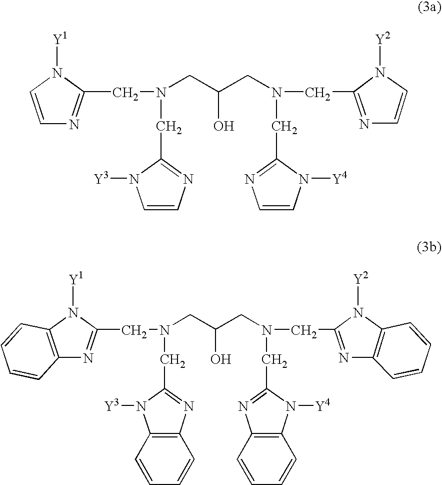
wherein each of Y¹, Y², Y³ and Y⁴ independently denotes a hydrogen atom, an alkyl group having 1 to 50 carbon atoms, or an aromatic group having 2 to 60 carbon atoms, and at least one of Y¹, Y², Y³ and Y⁴ is an alkyl group having 1 to 50 carbon atoms which has a polymerizable functional group, or an aromatic group having 2 to 60 carbon atoms which has a polymerizable functional group.

27. The complex monomer according to claim 22, having a structure in which L⁰² in the general formula (2-1) is expressed by the following general formula (40):
G⁰¹-(OCH₂CH(R))ₙOR (40)
wherein R denotes a hydrogen atom, an alkyl group having 1 to 50 carbon atoms which may have a substituent, or an aryl group having 6 to 50 carbon atoms which may have a substituent, and G⁰¹ denotes a substituent containing a functional group expressed by any of the following general formulas (4-1), (4-2), (4-3) and (4-4), respectively:

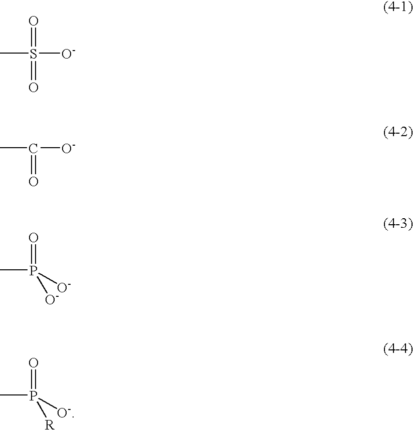

28. A polymer complex obtained by polymerizing the complex monomer described in claim 19.

29. A polymer complex obtained by copolymerizing the complex monomer described in claim 19 and a comonomer.

30. A redox catalyst, comprising the modified polymer complex described in claim 1.

31. A redox catalyst, comprising the complex monomer described in claim 19.

32. A redox catalyst, comprising the polymer complex described in claim 28.
